(19) United States
(12) Patent Application Publication
(10) Pub. No.: US 2020/0323428 A1
(43) Pub. Date: Oct. 15, 2020

(54) OPHTHALMIC APPARATUS

(71) Applicant: CANON KABUSHIKI KAISHA, Tokyo (JP)

(72) Inventor: Hiroto Tachikawa, Sagamihara-shi (JP)

(21) Appl. No.: 16/843,176

(22) Filed: Apr. 8, 2020

(30) Foreign Application Priority Data
Apr. 15, 2019 (JP) 2019-076800
Sep. 18, 2019 (JP) 2019-169513
Mar. 26, 2020 (JP) 2020-055559

(51) Int. Cl. A61B 3/00; A61B 3/10

(52) U.S. Cl. CPC A61B 3/0075; A61B 3/102; A61B 3/0083

(57) ABSTRACT

Provided is an ophthalmic apparatus including: an optical head portion including an optical system arranged to measure an eye to be inspected; a movable portion arranged to move the optical head portion in a horizontal direction relative to a base portion; an operation stick, which includes an urging portion arranged to apply an urging force between the base portion and the movable portion, and is provided on the movable portion so as to be tiltable; and an urging force reduction mechanism arranged to reduce the urging force when the operation stick is tilted by a predetermined angle or larger.

### BACKGROUND OF THE INVENTION

### Field of the Invention

The present invention relates to an ophthalmic apparatus.

### Description of the Related Art

In some cases, an ophthalmic apparatus includes an operation stick to be used for positioning an optical head portion, which is configured to measure an eye to be inspected, with respect to an eye to be inspected. At the time of performing fine positioning of finely positioning the optical head portion forward, backward, rightward, and leftward, a user such as an inspector performs an operation of tilting the operation stick provided to a movable portion. When the operation of tilting the operation stick is performed, a friction portion provided at a lower portion of the operation stick rolls on a surface of a friction plate provided below the friction portion, and a movable base supporting the optical head portion moves in the tilting direction of the operation stick.

Meanwhile, at the time of rough positioning of quickly moving the optical head portion in a horizontal direction by a large movement amount, the user grips the operation stick and, as needed, a part of the movable base and moves the movable base in the horizontal direction. With this, the friction portion provided at a lower end portion of the operation stick slides on the surface of the friction plate, and the movable portion moves in the horizontal direction relative to a base portion.

When a friction force generated between the friction portion, which is provided at the lower end portion of the operation stick, and the friction plate is small, at the time of fine positioning, slipping occurs between the lower portion of the operation stick and the friction plate. As a result, it is difficult to perform fine positioning. Meanwhile, when the friction force is large, at the time of rough positioning, a force required for moving the movable portion becomes larger. As a result, more effort is required for the user. As described above, the fine positioning and the rough positioning have conflicting requirements with regard to the friction force generated between the lower end portion of the operation stick and the friction plate.

In view of the circumstances described above, in Japanese Patent Application Laid-Open No. 2017-23410, the following configuration is disclosed. In order to reduce the force required for moving the movable portion at the time of rough positioning, a switching unit configured to switch between a contact state and a separated state of the friction portion and the friction plate is provided. However, with the configuration described in Japanese Patent Application Laid-Open No. 2017-23410, a user such as an inspector operates the switching unit, and hence work to be performed with a hand not holding the operation stick, such as work of operating the optical head portion for focus adjustment of an optical system or lifting up an eyelid of a subject to be inspected, is interrupted. Therefore, a degree of freedom in an operation for measuring an eye to be inspected is impaired. As a result, operational feeling in the rough positioning is degraded.

### SUMMARY OF THE INVENTION

The present invention has an object to provide an ophthalmic apparatus, which is capable of allowing a user to perform operations of positioning while consistently holding an operation stick by one hand at the time of rough positioning and fine positioning and is capable of reducing a force required for moving a movable portion at the time of rough positioning.

According to at least one embodiment of the present invention, there is provided an ophthalmic apparatus including: an optical head portion including an optical system arranged to measure an eye to be inspected; a movable portion arranged to move the optical head portion in a horizontal direction relative to a base portion; an operation stick, which includes an urging portion arranged to apply an urging force between the base portion and the movable portion, and is provided on the movable portion so as to be tiltable; and an urging force reduction mechanism arranged to reduce the urging force when the operation stick is tilted by a predetermined angle or larger.

Further features of the present invention will become apparent from the following description of exemplary embodiments with reference to the attached drawings.

### BRIEF DESCRIPTION OF THE DRAWINGS

FIG. 1A, FIG. 1B and FIG. 1C are views for illustrating a schematic configuration example of an ophthalmic apparatus according to a first embodiment of the present invention.

FIG. 2A and FIG. 2B are views for illustrating cross sections around an operation stick of the ophthalmic apparatus according to the first embodiment.

FIG. 3 is a view for illustrating a state in which the operation stick of the first embodiment is tilted.

FIG. 4 is a view for illustrating a state in which the operation stick of the first embodiment has reached a limit of a tilt angle.

FIG. 5 is a view for illustrating a cross section around an operation stick of an ophthalmic apparatus according to a second embodiment of the present invention.

FIG. 6 is a view for illustrating a friction portion of the second embodiment at the time of rough positioning.

FIG. 7 is a view for illustrating a cross section around an operation stick of an ophthalmic apparatus according to a third embodiment of the present invention.

FIG. 8 is an explanatory view for illustrating a friction portion of the third embodiment.

FIG. 9 is a view for illustrating a state in which the operation stick of the third embodiment is tilted.

FIG. 10A is a view for illustrating a cross section around an operation stick of a first modification example.

FIG. 10B is an enlarged cross-sectional view of another example of the operation stick of the first modification example.

FIG. 11 is a view for illustrating an example of a configuration of an ophthalmic apparatus according to a fourth embodiment of the present invention.

FIG. 12 is a view for illustrating an example of a configuration of a horizontal movement mechanism.

FIG. 13A and FIG. 13B are views for illustrating an example of a configuration of an inner ball of the fourth embodiment.

FIG. 14 is a view for illustrating an example of a state in which an operation stick is tilted.

FIG. 15A and FIG. 15B are views for illustrating an example of a configuration of an inner ball of a fifth embodiment of the present invention.

FIG. 16 is a view for illustrating an example of a configuration of an ophthalmic apparatus of a second modification example.

FIG. 17A is an explanatory view for schematically illustrating an operation of the operation stick according to the first embodiment of the present invention.

FIG. 17B is an explanatory view for schematically illustrating an operation of the operation stick according to the first embodiment of the present invention.

FIG. 17C is an explanatory view for schematically illustrating an operation of the operation stick according to the first embodiment of the present invention.

### DESCRIPTION OF THE EMBODIMENTS

Preferred embodiments of the present invention will now be described in detail in accordance with the accompanying drawings.

Matters described in the following embodiment, which include dimensions, materials, shapes or relative positions of components, are freely set, and can be changed depending on various conditions or configurations of apparatus to which the present invention is applied. In the drawings, the same reference symbols are used to denote components that are the same as one another or functionally similar to one another among the drawings.

For convenience of description, with regard to an ophthalmic apparatus according to each of the embodiments of the present invention, a right-and-left direction as seen from a subject to be inspected is represented as an X axis direction, an up-and-down direction is represented as a Y axis direction, and a front-and-back direction is represented as a Z axis direction. With regard to the up-and-down direction, an upward direction is represented as a +Y axis direction, and a downward direction is represented as a −Y axis direction. Moreover, “rough positioning” is defined as positioning of quickly moving an optical head in a horizontal direction by a large movement amount, and “fine positioning” is defined as positioning of finely moving the optical head in the horizontal direction by a smaller movement amount as compared to the rough positioning.

### First Embodiment

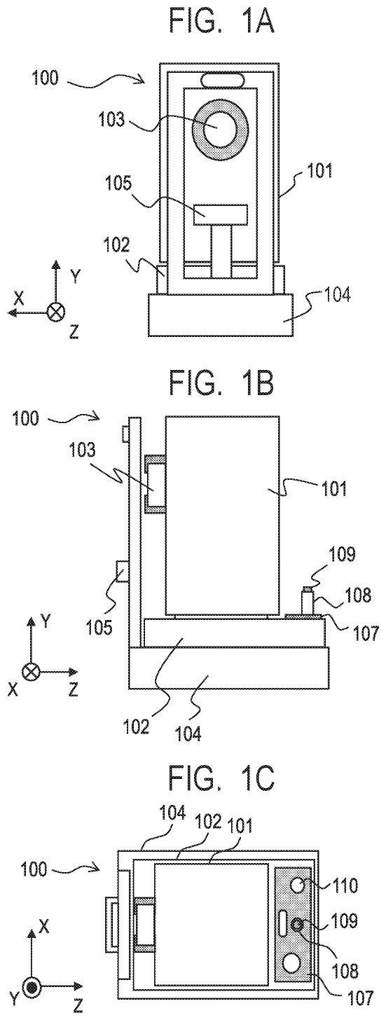

Here, with reference to FIG. 1A to FIG. 4, an ophthalmic apparatus according to a first embodiment of the present invention, in particular, an ophthalmic apparatus including an operation stick to be used for positioning with respect to an eye to be inspected is described. First, with reference to FIG. 1A to FIG. 1C, an overall configuration of the ophthalmic apparatus according to the first embodiment is described.

<Configuration of Ophthalmic Apparatus>

FIG. 1A to FIG. 1C are views for schematically illustrating an example of an overall configuration of an ophthalmic apparatus 100 according to the first embodiment. FIG. 1A is a view for illustrating the ophthalmic apparatus 100 as seen from a −Z direction. FIG. 1B is a view for illustrating the ophthalmic apparatus 100 as seen from a −X direction. FIG. 1C is a view for illustrating the ophthalmic apparatus 100 as seen from a +Y direction.

As illustrated in FIG. 1A to FIG. 1C, the ophthalmic apparatus 100 according to the first embodiment includes a base portion 104, a movable portion 102, and an optical head portion 101. The base portion 104 serves as a base for the ophthalmic apparatus 100. The base portion 104 includes a chin receiving part 105 arranged to receive a chin of a subject to be inspected.

The movable portion 102 is arranged to support the optical head portion 101 so that the optical head portion 101 can be raised and lowered in the Y axis direction. Moreover, the movable portion 102 is provided on the base portion 104 through intermediation of a horizontal movement mechanism, which is described later, so that the movable portion 102 is movable in an X-Z axis direction being a horizontal direction. When the movable portion 102 moves in the X-Z axis direction relative to the base portion 104, the optical head portion 101 also moves in the X-Z axis direction relative to the base portion 104 integrally with the movable portion 102. In order to enable the movable portion 102 to move in the X-Z axis direction relative to the base portion 104, the movable portion 102 and the base portion 104 are retained in a state of having a predetermined clearance in the Y axis direction.

Moreover, the ophthalmic apparatus 100 includes a raising and lowering mechanism arranged to raise and lower the optical head portion 101 in the Y axis direction. For example, a feed screw mechanism is applied to the raising and lowering mechanism. For example, a screw shaft is mounted to the movable portion 102 so that the screw shaft is rotatable about an axis thereof directed in a direction parallel to the Y axis direction, and a nut which is threadedly engaged with the screw shaft is mounted to the optical head portion 101. When the screw shaft rotates, the optical head portion 101 is raised or lowered in the Y axis direction relative to the movable portion 102 together with the nut. A configuration for rotating the screw shaft is described later.

The optical head portion 101 is arranged to measure an eye to be inspected, and various optical systems arranged to measure an eye to be inspected are provided to the optical head portion 101. For example, the optical head portion 101 includes an object lens portion 103, an OCT unit (not shown), an SLO unit (not shown), an anterior-eye observation unit (not shown), and a fixation lamp unit (not shown). The OCT unit includes an optical system adapted to optical coherence tomography (OCT). Moreover, the SLO unit includes an optical system adapted to a scanning laser ophthalmoscope (SLO).

The object lens portion 103 is arranged to emit laser to an eye to be inspected (not shown) and obtain scattered light having returned from the eye to be inspected. The OCT unit and the SLO unit are arranged to obtain a tomographic image and a fundus image of an eye to be inspected based on scattered light information of laser emitted from the object lens portion 103, respectively. The anterior-eye observation unit is arranged to image an anterior eye portion of an eye to be inspected and display an anterior eye portion image on a display portion such as a monitor (not shown). A user such as an inspector sees the anterior eye portion image displayed on the display portion and positions the optical head portion 101 with respect to the eye to be inspected. The fixation lamp unit displays indicators such as “x” and “∘” (not shown) to lead a fixation direction of the eye to be inspected.

The optical head portion 101 is only required to have a configuration in which an optical system for measuring an eye to be inspected is provided, and a specific configuration of the optical head portion 101 is not limited. A configuration having hitherto been publicly known may be applied to the optical head portion 101. Moreover, a configuration of the optical system provided to the optical head portion 101 and items to be measured are also not particularly limited.

An operation stick 108 and an operation panel 107 are provided to the movable portion 102. The operation stick 108 is provided so as to be tiltable in a freely selected direction relative to the movable portion 102. An imaging button 109, which is an electrical switch, is provided on the top of the operation stick 108. A controller (information processing device) (not shown) connected to the base portion 104 starts measuring (for example, imaging) an eye to be inspected through use of the optical head portion 101 when an operation of the imaging button 109 is detected.

Moreover, a lock button 110 is provided to the movable portion 102. The lock button 110 is an operation member to be used for operating a locking mechanism arranged to lock the movement of the movable portion 102 in the X-Z axis direction. The locking mechanism is an example of a fixing unit arranged to fix the movable portion 102 so that the movable portion 102 cannot move relative to the base portion 104. When the lock button 110 is pressed, the movable portion 102 is locked on the base portion 104 by the locking mechanism so that the movable portion 102 does not move in the X-Z axis direction relative to the base portion 104. When the lock button 110 is pressed again in this state, the locking of the movable portion 102 is released so that the movable portion 102 is movable in the X-Z axis direction relative to the base portion 104. As described above, the lock button 110 is a so-called knock-type mechanism. A configuration having hitherto been publicly known may be applied to the locking mechanism for locking the movable portion 102 on the base portion 104.

Next, with reference to FIG. 2A and FIG. 2B, the movable portion 102, the base portion 104, and the operation stick 108 of the ophthalmic apparatus 100 are described. FIG. 2A and FIG. 2B are views for schematically illustrating an example of a sectional configuration of an internal structure of the movable portion 102, the base portion 104, and the operation stick 108 of the ophthalmic apparatus 100. FIG. 2A is a sectional view taken along a plane extending along a center of the imaging button 109 of the operation stick 108 and being orthogonal to the X axis. FIG. 2B is a sectional view taken along a plane extending along the center of the imaging button 109 of the operation stick 108 and being orthogonal to the Z axis.

The ophthalmic apparatus 100 includes the horizontal movement mechanism arranged to move the movable portion 102 in the horizontal direction. For example, the movable portion 102 may include a first plurality of guides for moving the movable portion 102 in a first direction of the horizontal direction, and a second plurality of guides for moving the movable portion 102 in a second direction of the horizontal direction, which intersects with the first direction. In the first embodiment, as illustrated in FIG. 2A, linear cross roller guides 219 and linear cross roller guides 220 are applied as an example of the horizontal movement mechanism. The linear cross roller guides 219 are arranged to move the movable portion 102 in the X axis direction. The linear cross roller guides 220 are arranged to move the movable portion 102 in the Z axis direction. The linear cross roller guides 219 and 220 are each a linear-motion guiding mechanism in which cylindrical rollers having a retainer are incorporated between two raceway bases each having a V-shaped raceway groove with two flat surfaces (not shown). Those linear cross roller guides 219 and 220 are provided so as to cross one another, and the base portion 104 and the movable portion 102 are coupled to each other by the linear cross roller guides 219 and 220. The linear cross roller guides 219 and 220 are only required to be provided in directions intersecting each other. Moreover, the guiding mechanism is not limited to the cross-roller guides, and may be freely selected guides.

With such a configuration, the movable portion 102 is freely movable in the X-Z axis direction being the horizontal direction. Therefore, the optical head portion 101 can be positioned in the X-Z axis direction with respect to an eye to be inspected. Moreover, through the use of the horizontal movement mechanism including the plurality of guides, such as the linear cross roller guides, arranged in directions intersecting each other in the horizontal direction, the weight of the optical head portion 101 and the weight of the movable portion 102 can be supported. Therefore, the weight of the optical head portion 101 and the weight of the movable portion 102 can be prevented from being included in an urging force (biasing force), which is described below, applied between a lower end portion (friction portion 223) of the operation stick 108 and the base portion 104 (friction plate 222). The ophthalmic apparatus 100 includes the horizontal movement mechanism which enables the optical head portion 101 to move in the X-Z axis direction, and hence the base portion 104 and the movable portion 102 are retained in a state of having a predetermined clearance in the Y axis direction. Moreover, it is only required that the movable portion 102 be movable in the X-Z axis direction (horizontal direction) relative to the base portion 104, and a specific configuration of the horizontal movement mechanism is not limited to the configuration described above. A publicly known two-dimensional linear stage may be applied to the horizontal movement mechanism.

A movable-portion base 201 serves as a base for the movable portion 102. An operation stick supporting portion 202 is arranged to support the operation stick 108 so that the operation stick 108 is tiltable, and is fixed to the movable-portion base 201 by, for example, a screw (not shown). For example, a plate metal member is applied to the operation stick supporting portion 202.

An inner-ball base 203 (inner-ball accommodating portion) is a hollow member mounted to the operation stick supporting portion 202, and an inner ball 205 is accommodated inside the inner-ball base 203. The inner ball 205 has a spherical shape. At a center of the inner ball 205, a through hole for allowing a center shaft 206 of the operation stick 108 to pass therethrough is formed. In an outer peripheral surface of the inner ball 205, a slit groove 235 to which a rotation stop pin 204 is fitted as illustrated in FIG. 2B is formed. At the time of the operation of tilting the operation stick 108, slip resistance is generated between the outer peripheral surface of the inner ball 205 and an inner peripheral surface of the inner-ball base 203.

The center shaft 206 of the operation stick 108 is a hollow shaft. The center shaft 206 is inserted through the through hole formed in the inner ball 205 and is reciprocally movable in an axial direction of the center shaft 206. A cutout portion 227 having an inverted U-shape is formed at a lower end portion of the center shaft 206. The cutout portion 227 is formed to allow an electric harness 299, which is connected to the imaging button 109, to pass therethrough.

An elongated hole 207 is formed at an intermediate portion of the center shaft 206 in a longitudinal direction of the center shaft 206 as illustrated in FIG. 2B. The elongated hole 207 is a through hole which allows communication between an inner periphery side and an outer periphery side of the center shaft 206 and is an elongated hole extending long in the longitudinal direction of the center shaft 206. A regulation pin 208 (regulation member) is provided to the operation stick 108. As illustrated in FIG. 2A, the regulation pin 208 passes through a screw hole 210 (hole portion) of the inner ball 205 and the elongated hole 207 (hole portion) of the center shaft 206. The regulation pin 208 is, for example, a screw having a distal end formed into a pin. With the regulation pin 208 and the elongated hole 207, a range of the reciprocal movement of the center shaft 206 in the axial direction relative to the inner ball 205 is regulated.

A friction portion 223 is fixed at the lower end portion of the center shaft 206 of the operation stick 108 by a screw (not shown). A lower surface of the friction portion 223 has a spherical surface shape protruding toward a lower surface side (that is, toward a friction plate 222 provided to the base portion 104). A spherical surface radius is substantially equal to a distance between the center of the inner ball 205 and an upper surface of the friction plate 222, and a movement resolution of the fine positioning (movement amount of the optical head portion 101 in accordance with a tilt angle of the operation stick 108) described later is determined based on the spherical surface radius.

A standing wall 236 having a cylindrical shape for fitting the friction portion 223 to the center shaft 206 is provided on an upper surface side of the friction portion 223 as illustrated in FIG. 2B, and a cutout portion 237 having a substantially U-shape is formed in the standing wall 236. A position of the cutout portion 227 of the center shaft 206 and a position of the cutout portion 237 of the standing wall 236 of the friction portion 223 match each other in a circumferential direction of the center shaft 206 and the friction portion 223. Therefore, the electric harness 299 of the imaging button 109 can be drawn to the outside from the center shaft 206 through the cutout portions 227 and 237.

A coil spring 224 (urging member or biasing member) is provided between the friction portion 223 and the inner ball 205. The inner ball 205 is urged to be brought into contact with the inner peripheral surface of the inner-ball base 203 by an urging force (biasing force) of the coil spring 224, and the friction portion 223 (in particular, a portion which corresponds to a lower surface of the friction portion 223 and is formed into the spherical surface shape) is urged to be brought into contact with the friction plate 222. With this, the urging force is applied between the base portion 104 and the movable portion 102. Therefore, the coil spring 224 functions as an example of an urging portion (biasing portion) arranged to apply the urging force between the base portion 104 and the movable portion 102. Inner ball 205 constructs a part of the operation stick 108, and the inner-ball base 203 constructs a part of the movable portion 102.

In the fine positioning, a user tilts the operation stick 108 to move the optical head portion 101 by the movement of the movable portion 102 in accordance with the tilt of the operation stick 108. At this time, a friction force generated between the friction portion 223 and the friction plate 222 with respect to the urging force between the base portion 104 and the movable portion 102 prevents occurrence of slipping between the friction portion 223 and the friction plate 222, thereby being capable of easily performing the fine positioning. In this regard, when a tilt angle of the operation stick 108 falls within the range of from 0 degrees (upright state) to a predetermined angle, the tilt of the operation stick 108 and the friction force generated between the friction portion 223 and the friction plate 222 with respect to the urging force between the base portion 104 and the movable portion 102 cause the movable portion 102 to move in the horizontal direction. The optical head portion 101 is movable in the horizontal direction relative to the base portion 104 by the movement of the movable portion 102.

As described above, the urging force between the base portion 104 and the movable portion 102 as well as a rolling friction force generated between the friction plate 222 and the friction portion 223 are used for moving the movable portion 102 in the fine positioning. Meanwhile, the friction force generated between the friction plate 222 and the friction portion 223 may be resistance against an operation at the time of rough positioning. Moreover, even when a user releases the operation stick 108, owing to the friction force generated between the inner ball 205 and the inner-ball base 203, the movable portion 102 can be retained so as to be prevented from being moved by a reaction force of a bundle of the electric harness 299 wired from the base portion 104 to the movable portion 102.

The rotation stop pin 204 illustrated in FIG. 2B is a screw having a distal end formed into a pin. The rotation stop pin 204 is screwed to the inner-ball base 203 and projects toward the center of the inner ball 205 to be fitted to the slit groove 235 of the inner ball 205. Therefore, the rotation of the center shaft 206 relative to the movable portion 102 is regulated, and only the tilting is allowed. When the rotation of the center shaft 206 is regulated, damage on the electric harness 299 such as an electric harness of the imaging button 109 arranged inside the center shaft 206 can be prevented.

A relay member 209 is arranged to fix the imaging button 109 to the center shaft 206 and is fixed on the top of the center shaft 206 by a screw (not shown). Exterior covers 212 to 214 of the operation stick 108 are arranged so as to cover the center shaft 206 and the relay member 209 of the operation stick 108 and are rotatable coaxially with the center shaft 206 of the operation stick 108.

Specifically, the following structure is given. Deep groove ball bearings 228 and 229 are provided between the center shaft 206 and the exterior cover 214. Inner rings of the deep groove ball bearings 228 and 229 are fitted to the center shaft 206, and outer rings of the deep groove ball bearings 228 and 229 are fitted to the exterior cover 214. The inner ring of the deep groove ball bearing 228 is held in abutment against a protruding portion 231 provided on the center shaft 206, and the outer ring of the deep groove ball bearing 228 is held in abutment against an abutment portion 232 of the exterior cover 214. Moreover, the inner ring of the deep groove ball bearing 229 is held in abutment against an abutment portion 298 of the relay member 209, and the outer ring of the deep groove ball bearing 229 is held in abutment against the abutment portion 232 of the exterior cover 214. With this, the exterior cover 214 is positioned with respect to the center shaft 206 in the axial direction. Other exterior covers 212 and 213 are fixed to the exterior cover 214 by bonding or by a screw (not shown).

With such a configuration, the exterior covers 212 to 214 are rotatable relative to the center shaft 206. It is only required that the exterior covers 212 to 214 be coaxially rotatable in a state of being positioned with respect to the center shaft 206 in the axial direction, and a coupling structure between the exterior cover 214 and the center shaft 206 is not limited to the configuration described above.

An annular member 238 is fixed to the center shaft 206 by a screw (not shown). The annular member 238 is provided below the protruding portion 231 and above the inner ball 205 so that, in an operation of the rough positioning described later, the annular member 238 is brought into abutment against an upper surface 239 of the inner-ball base 203 when the operation stick 108 is tilted by a predetermined angle. It is not always required that the annular member 238 and the center shaft 206 be separate members. For example, the protruding portion 231 provided on the center shaft 206 may serve also as the annular member 238.

Here, with reference to FIG. 2A, a configuration of the raising and lowering mechanism arranged to raise and lower the optical head portion 101 in the Y axis direction is described. As described above, the exterior covers 212 to 214 of the operation stick 108 are arranged so as to be rotatable coaxially with the center shaft 206. A pulley gear 216 which rotates together with the exterior covers 212 to 214 are provided to the ophthalmic apparatus 100, and a pulley belt 218 is wound around the pulley gear 216 and the screw shaft of the feed screw mechanism arranged to raise and lower the optical head portion 101 in the Y axis direction. Therefore, when the exterior covers 212 to 214 of the operation stick 108 are rotated by a user, the rotation is transmitted to the screw shaft via the pulley gear 216 and the pulley belt 218, and the optical head portion 101 is raised and lowered in the Y axis direction.

Specifically, the pulley gear 216 is provided coaxially with the inner-ball base 203, and a deep groove ball bearing 215 is provided between the pulley gear 216 and the inner-ball base 203. An outer ring of the deep groove ball bearing 215 is fitted to the pulley gear 216, and an inner ring of the deep groove bearing 215 is fitted to the inner-ball base 203. Therefore, the pulley gear 216 is rotatable relative to the inner-ball base 203.

Further, a pin screw 217 is fixed to the pulley gear 216. The pin screw 217 is a screw having a distal end portion formed into a pin shape and projects toward the exterior cover 214 so that an axis of the pin screw 217 intersects the center of the inner ball 205. A slit groove 234 is formed in an outer peripheral surface of the exterior cover 214, and the distal end portion of the pin screw 217 is fitted to the slit groove 234. There is given a relationship in which an outer diameter of the distal end portion of the pin screw 217 conforms to a width of the slit groove 234. The pulley belt 218 is wound around the pulley gear 216 and the screw shaft of the feed screw mechanism (not shown) arranged to raise and lower the optical head portion 101 in the Y axis direction.

With such a configuration, when the exterior covers 212 to 214 of the operation stick 108 are rotated by a user, the rotation is transmitted to the screw shaft described above via the pulley gear 216 and the pulley belt 218. When the screw shaft rotates, a nut which is threadedly engaged with the screw shaft is raised and lowered, and the optical head portion 101 is raised and lowered together with the nut in the Y axis direction. As described above, when the exterior covers 212 to 214 of the operation stick 108 are rotated by a user, the optical head portion 101 is raised and lowered in the Y axis direction, thereby being capable of positioning the optical head portion 101 with respect to an eye to be inspected.

A configuration of the raising and lowering mechanism is not limited to the configuration described above. Any publicly known mechanism which is capable of raising and lowering the optical head portion 101 in the Y direction in accordance with a freely selected operation such as a rotational operation on the operation stick 108 may be applied to the raising and lowering mechanism.

<Operation of Fine Positioning>

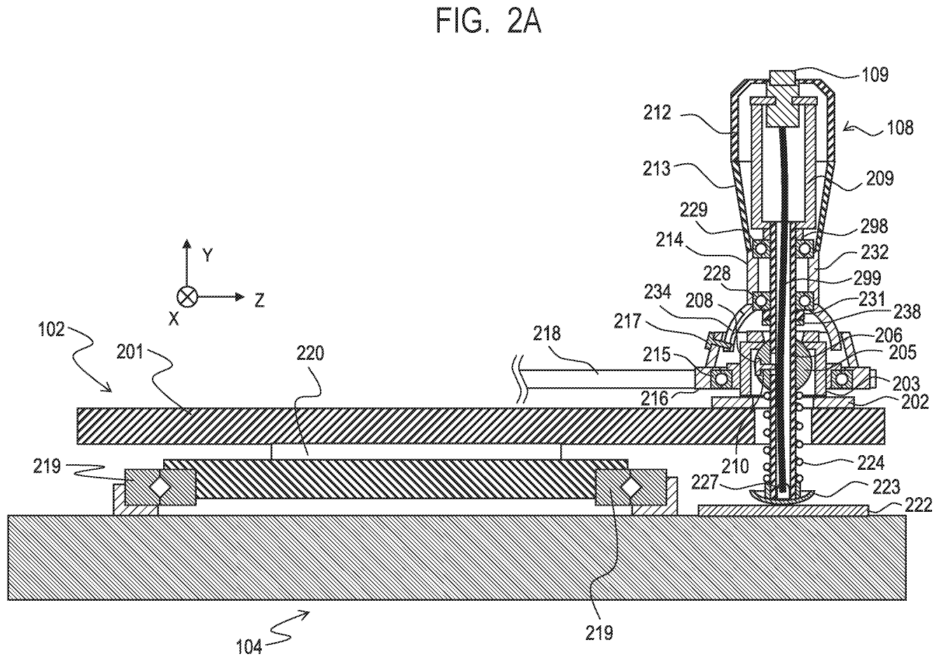

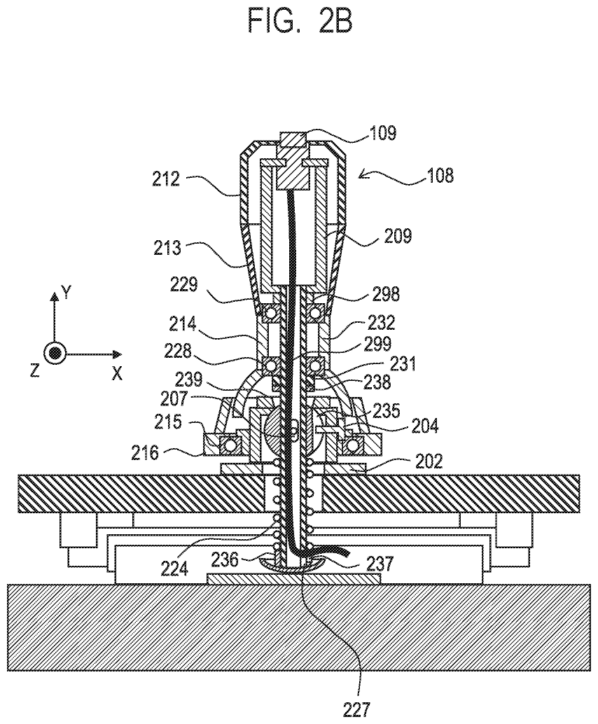

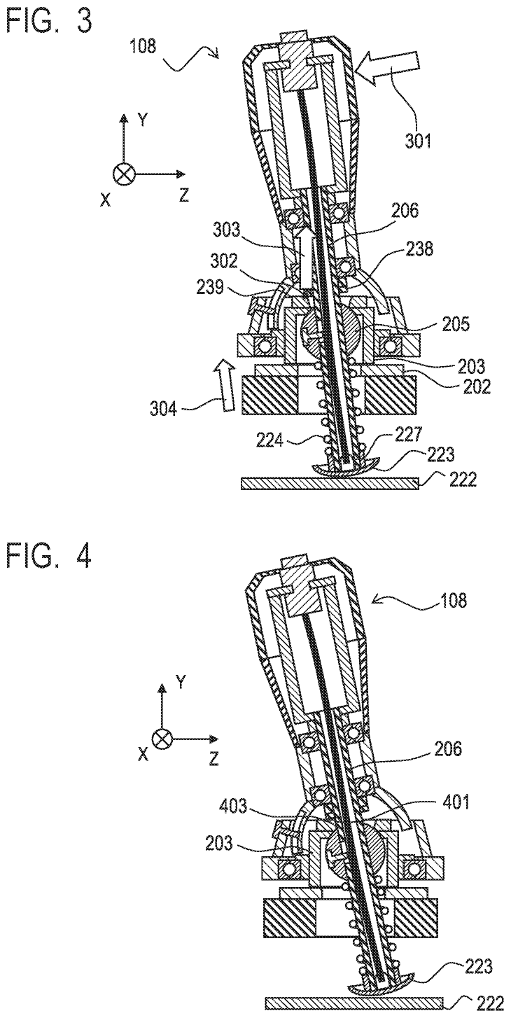

Here, an operation of the fine positioning of the optical head portion 101 in the X-Z direction (horizontal direction) is described. The operation stick 108 is tilted by a user such as an inspector in a direction of moving the optical head portion 101. When the operation stick 108 is tilted, the friction portion 223 rolls on the surface of the friction plate 222 without slipping. The optical head portion 101 moves substantially in proportional to a tilt angle of the operation stick 108 until the optical head portion 101 reaches the state of FIG. 3 described later.

<Operation of Rough Positioning>

Next, with reference to FIG. 3 and FIG. 4, an operation of the rough positioning of the optical head portion 101 in the X-Z direction (horizontal direction) is described. FIG. 3 is a view for illustrating a state in which the operation stick 108 is tilted by a user at the time of rough positioning.

In the case of the rough positioning of the optical head portion 101, under a state in which the exterior cover 212 of the operation stick 108 is held by a user such as an inspector, the operation stick 108 is tilted in a direction of moving the optical head portion 101. At this time, the exterior cover 212 receives an operation force 301 applied by a user to tilt the operation stick 108. When the operation stick 108 is tilted, a corner portion of the annular member 238 is brought into contact with a contact point 302 on the upper surface 239 of the inner-ball base 203 as illustrated in FIG. 3. At the contact point 302, the annular member 238 receives a reaction force 303 substantially in the +Y direction (upward) from the inner-ball base 203.

As described above, the annular member 238 and the center shaft 206 are fixed to each other by a screw (not shown), and the center shaft 206 and the friction portion 223 are fixed to each other by a screw (not shown). Therefore, the friction portion 223 receives a force in the axial direction 304 of the center shaft 206 by the reaction force 303. With this, an urging force generated between the friction portion 223 and the friction plate 222 is reduced, and the friction force generated between the friction portion 223 and the friction plate 222 is reduced. Therefore, the annular member 238 functions as an example of an urging force reduction mechanism (biasing force reduction mechanism) arranged to reduce the urging force generated between the friction portion 223 and the friction plate 222 to reduce the urging force generated between the base portion 104 and the movable portion 102.

Here, when the operation stick 108 is tilted by the predetermined angle or larger, the urging force reduction mechanism is capable of reducing the urging force between the base portion 104 and the movable portion 102 based on the reaction force 303 at the movable portion 102 against the operation force 301 being applied to the operation stick 108. More specifically, the urging force reduction mechanism is capable of reducing the urging force between the base portion 104 and the movable portion 102 through use of the force in the axial direction 304 of the operation stick 108 generated by the reaction force 303.

When the friction force generated between the friction portion 223 and the friction plate 222 is reduced by reducing the urging force generated between the friction portion 223 and the friction plate 222, the operation force for the rough positioning is reduced as compared to a state in which the urging force generated between the friction portion 223 and the friction plate 222 is not reduced. As a result, a force required for moving the optical head portion 101 can be reduced, and hence a user can move the optical head portion 101 with a small operation force.

Here, when the operation stick 108 is tilted to an angle larger than the predetermined angle, while the annular member 238 slides on the upper surface 239 of the inner-ball base 203, the operation stick 108 tilts about a contact point between the annular member 238 and the upper surface 239 as a center (fulcrum). At this time, the tilt of the operation stick 108 about the contact point between the annular member 238 and the upper surface 239 as the center allows the center shaft 206 of the operation stick 108 to move in a direction of separating away from the friction plate 222 in the axial direction relative to the inner ball 205 accommodated in the inner-ball base 203 of the movable portion 102. In other words, when the operation stick 108 is tilted by the predetermined angle or larger, the shaft portion (center shaft 206) of the operation stick 108 moves in the axial direction relative to the movable portion 102 so that a distance between a lower portion (friction portion 223) of the operation stick 108 and the movable portion 102 in the axial direction of the operation stick 108 becomes shorter than the distance given when a tilt angle of the operation stick 108 falls within the range of from 0 degrees to the predetermined angle. With this, the urging force reduction mechanism is capable of reducing the urging force between the base portion 104 and the movable portion 102.

Further, FIG. 4 is a view for illustrating a state in which the operation stick 108 has reached a limit (upper limit) of the tilt angle. When the operation stick 108 is further tilted by a user from the state of FIG. 3, an abutment portion 401 of the center shaft 206 is brought into abutment against an angle regulation surface 403 of the inner-ball base 203. This state corresponds to the limit of the tilt angle of the operation stick 108. Here, the limit of the tilt angle corresponds to an angle which causes a force in a direction opposite to an operation force being applied to the operation stick 108 in a tilting direction of the operation stick 108 to act on the operation stick 108. More specifically, the limit of the tilt angle corresponds to an angle at which the tilt of the operation stick 108 is regulated by the abutment of the abutment portion 401 of the center shaft 206 against the angle regulation surface 403 of the inner-ball base 203.

When the operation stick 108 is tilted to such a tilt limit angle, the friction portion 223 is not contact with the friction plate 222 as illustrated in FIG. 4. With this, the urging force and the friction force generated between the friction portion 223 and the friction plate 222 is lost. Thus, the force required for moving the optical head portion 101 can be further reduced, and a user can move the optical head portion 101 with a smaller operation force.

In the first embodiment, the following configuration is given. Specifically, when the operation stick 108 is tilted to the tilt limit angle, the center shaft 206 of the operation stick 108 is supported by the angle regulation surface 403 (supporting portion) of the inner-ball base 203 so that the friction portion 223 is brought into non-contact with the friction plate 222. However, the tilt angle of the operation stick 108 at which the center shaft 206 is supported by the supporting portion of the inner-ball base 203 so that the friction portion 223 is brought into non-contact with the friction plate 222 is not limited to such an angle and may be a tilt angle smaller than the tilt limit angle. The tilt angle of the operation stick 108 at which the friction portion 223 is brought into non-contact with the friction plate 222 may be suitably set in accordance with a desired configuration.

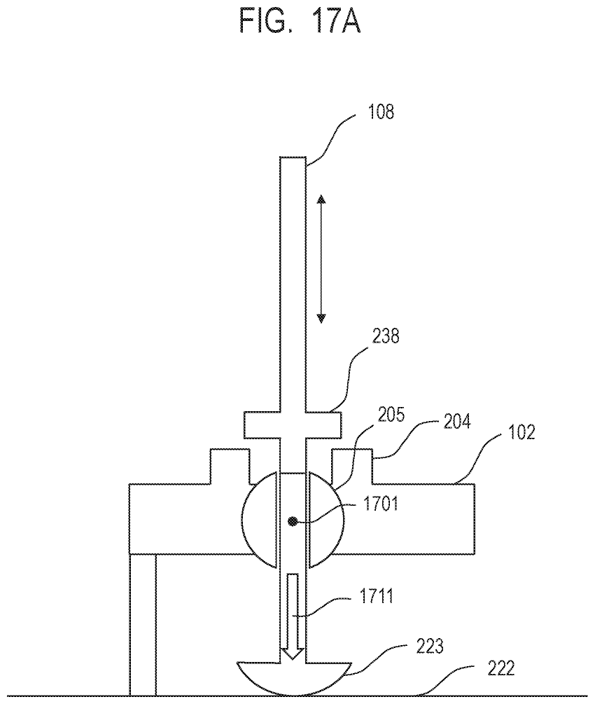

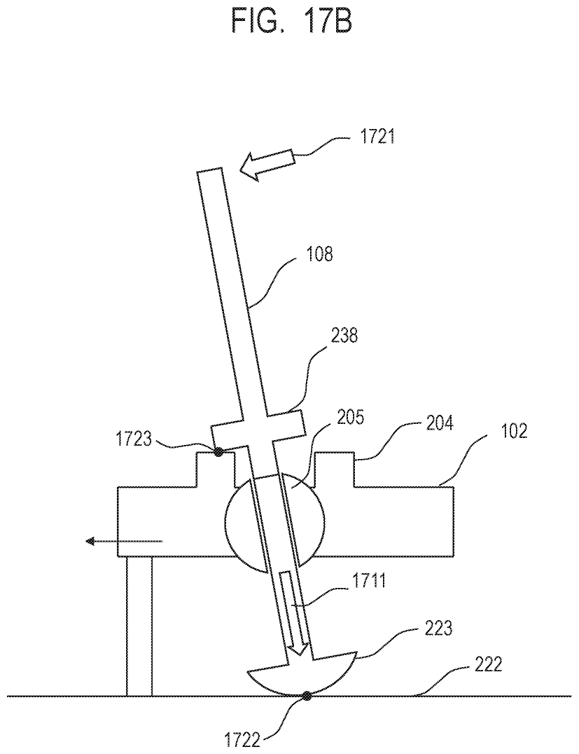

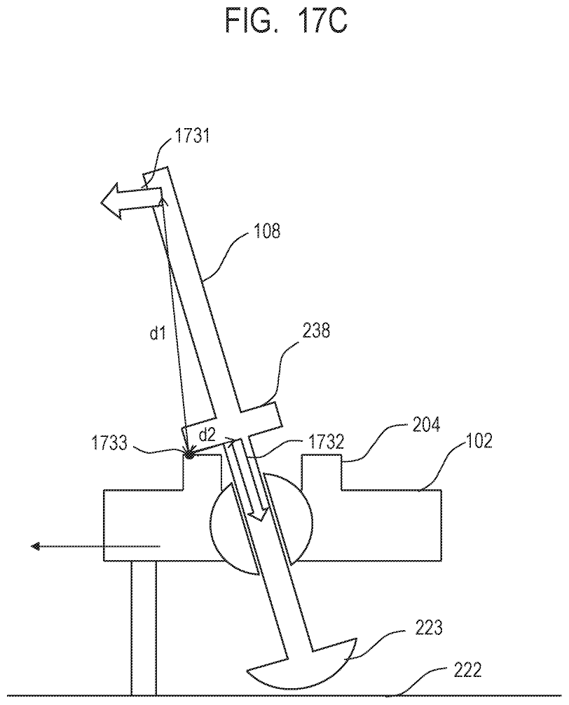

Next, an operation of the operation stick 108 according to the first embodiment is described with reference to FIG. 17A to FIG. 17C. FIG. 17A to FIG. 17C are explanatory views for schematically illustrating an operation of the operation stick 108 according to the first embodiment. FIG. 17A is an illustration of a state in which the operation stick 108 is not tilted. FIG. 17B is an illustration of a state in which the operation stick 108 is tilted to the predetermined angle at the time of fine positioning. FIG. 17C is an illustration of a state in which the operation stick 108 is tilted to an angle larger than the predetermined angle at the time of rough positioning. As illustrated in FIG. 17A, under the state in which the operation stick 108 is not tilted, the operation stick 108 is movable in the axial direction and is rotatable about a rotation center 1701 as a center. Moreover, as described above, the operation stick 108 receives an urging force (biasing force) 1711 from the coil spring 224 (urging member) so as to be urged toward the friction plate 222.

At the time of fine positioning, as illustrated in FIG. 17B, when an operation force 1721 is applied by a user, the operation stick 108 is tilted about a point 1722, at which the friction portion 223 being urged by the urging force 1711 is in contact with the friction plate 222, as a fulcrum. At this time, in accordance with the tilt of the operation stick 108, the movable portion 102 moves in the horizontal direction, and the optical head portion 101 being supported by the movable portion 102 moves. After that, when the operation stick 108 is tilted to the predetermined angle, the annular member 238 of the operation stick 108 is brought into contact with the inner-ball base 203 of the movable portion 102 at a contact point 1723.

Under the state in which the annular member 238 of the operation stick 108 and the inner-ball base 203 of the movable portion 102 are in contact with each other, when the operation stick 108 is tilted to the predetermined angle or larger, as illustrated in FIG. 17C, the operation stick 108 rotates about the contact point 1723 as a fulcrum 1733. Here, the operation force 1731 is a force in a direction of tilting the operation stick 108, and the urging force 1732 is a force in a direction of causing the operation stick 108 to stand upright. Therefore, it can be considered that the moment of the operation force 1731 and the moment of the urging force 1732 are moments in opposite directions. Therefore, the operation force 1731 causes a force in the axial direction of the operation stick 108 which is opposite to the direction of the urging force 1732 to be applied to the operation stick 108. Accordingly, when the operation stick 108 is tilted by the predetermined angle or larger, the urging force reduction mechanism including the annular member 238 is capable of reducing the urging force with the force in the axial direction of the operation stick 108 which is generated by the operation force applied to the operation stick 108 and by the movable portion 102. Here, consideration has been made of the state in which the moment of the operation force 1731 and the moment of the urging force 1732 are moments in opposite directions. However, in actuality, the operation force applied to the operation stick 108 by a user may include not only the operation force 1731 but also, for example, a force (force caused by a friction force generated between the outer peripheral surface of the inner ball 205 and the inner-ball base 203) balanced with the moment of the reaction force received by the center shaft 206 from the inner ball 205 and a force balanced with the friction force at the linear cross roller guides 219 and 220 at the time of tilting of the operation stick 108.

When the moment of the operation force 1731 is larger than the moment of the urging force 1732, the operation stick 108 is tilted to an angle larger than the predetermined angle. In this case, while the annular member 238 slides on the upper surface of the inner-ball base 203, the operation stick 108 tilts about the contact point between the annular member 238 and the upper surface 239 as the fulcrum 1733. As illustrated in FIG. 17C, the tilt (rotation) of the operation stick 108 about the fulcrum 1733 as the center causes the operation stick 108 to move in the direction of separating away from the friction plate 222 in the axial direction relative to the inner ball 205 accommodated in the inner-ball base 203 of the movable portion 102.

More specifically, when the force in the axial direction of the operation stick 108 generated by the moment of the operation force 1731 becomes larger than the urging force 1711 of the coil spring 224 (urging member) at the time of fine positioning, the operation stick 108 moves in the direction in which the length of the coil spring 224 is shortened in the axial direction. In the operation stick 108, at the time of fine positioning, the urging force 1711 of the coil spring 224 between the inner ball 205 and the friction portion 223 causes the friction portion 223 to be urged toward the friction plate 222, thereby bringing the friction portion 223 and the friction plate 222 into contact with each other. In contrast, when the operation stick 108 moves in the direction in which the length of the coil spring 224 becomes shorter than the length of the coil spring 224 at the time of fine positioning, the operation stick 108 is brought into a state in which the friction portion 223 is separated away from the friction plate 222.

In other words, when the operation stick 108 is tilted by the predetermined angle or larger, the shaft portion (center shaft 206) of the operation stick 108 moves in the axial direction relative to the movable portion 102 so that a distance between the lower portion (friction portion 223) of the operation stick 108 and the movable portion 102 in the axial direction of the operation stick 108 becomes shorter than the distance given when the tilt angle of the operation stick 108 falls within the range of from 0 degrees to the predetermined angle. With this, the friction portion 223 is separated away from the friction plate 222 so that the contact with the friction plate 222 is lost, and hence the urging force reduction mechanism is capable of further reducing the urging force between the base portion 104 and the movable portion 102.

When the operation stick 108 moves in the direction in which the length of the coil spring 224 becomes shorter than the length of the coils spring 224 at the time of fine positioning, the urging force of the coil spring 224 also becomes larger. In contrast, with the configuration described above, a distance d1 between a point of action to which the operation force 1731 is applied and the fulcrum 1733 can be set sufficiently longer than a distance d2 between the point of action to which the urging force 1732 is applied and the fulcrum 1733 (d1>>d2). Therefore, the operation force 1731 required for achieving the moment (F1×d1) of the operation force 1731 (F1) larger than the moment (F2×d2) of the urging force 1732 (F2) can be set to a small force. Thus, a user can reduce the urging force between the base portion 104 and the movable portion 102 with the operation force 1731 smaller than the force for moving the operation stick 108 in the axial direction, and can perform the rough positioning.

Moreover, when a user keeps applying the operation force 1731 to the operation stick 108 so that the moment of the operation force 1731 is balanced with the moment of the urging force 1732, the state in which the urging force is reduced can be maintained. Therefore, the state in which the force required for moving the optical head portion 101 at the time of rough positioning is reduced can be maintained. Here, as described above, the distance d1 can be set sufficiently longer than the distance d2. Therefore, the operation force required for achieving the moment (F1×d1) of the operation force 1731 (F1) balanced with the moment (F2×d2) of the urging force 1732 (F2) can be set to a smaller force. Thus, a user can perform the rough positioning with a smaller operation force.

As described above, the ophthalmic apparatus 100 according to the first embodiment includes the optical head portion 101 including the optical system for measuring an eye to be inspected, the movable portion 102 arranged to move the optical head portion 101 in the horizontal direction relative to the base portion 104, and the operation stick 108 provided on the movable portion 102 so as to be tiltable. Here, the operation stick 108 includes the urging portion arranged to apply the urging force between the base portion 104 and the movable portion 102. Moreover, the ophthalmic apparatus 100 includes the friction portion 223 provided at the lower end portion of the operation stick 108 and the friction plate 222 provided on the base portion 104 at a position below the friction portion 223. Further, the ophthalmic apparatus 100 includes the urging force reduction mechanism arranged to reduce the urging force generated by the urging portion between the movable portion 102 and the base portion 104 when the operation stick 108 is tilted by a predetermined angle or larger.

The urging portion is arranged to urge the friction portion 223 toward the friction plate 222. The urging portion includes the coil spring 224 (urging member) arranged to urge the inner ball 205 and the friction portion 223 provided to the operation stick 108 in the longitudinal direction of the operation stick 108. Moreover, the movable portion 102 includes the inner-ball base 203 arranged to accommodate the inner ball 205.

The urging force reduction mechanism is arranged to reduce the urging force generated between the movable portion 102 and the base portion 104 by reducing the urging force of the friction portion 223 against the friction plate 222 when the operation stick 108 is tilted by the predetermined angle or larger. More specifically, the urging force reduction mechanism includes the annular member 238 provided to the operation stick 108, and the annular member 238 is brought into contact with the inner-ball base 203 of the movable portion 102 when the operation stick 108 is tilted by the predetermined angle or larger. When the annular member 238 is brought into contact with the inner-ball base 203 of the movable portion 102, the reaction force against the operation force applied to the operation stick 108 is applied from the movable portion 102 to the operation stick 108. Then, the urging force of the friction portion 223 against the friction plate 222 is reduced based on the reaction force. The predetermined angle is an angle at which the operation stick 108 is brought into abutment against the movable portion 102 (brought into one-point contact), and the urging force between the movable portion 102 and the base portion 104 is reduced at an angle equal to or larger than the predetermined angle.

With this, the urging force generated between the movable portion 102 and the base portion 104 is reduced, and hence the friction force generated between the friction portion 223 and the friction plate 222 is reduced. Therefore, the operation force for the rough positioning is reduced as compared to the state in which the urging force generated between the movable portion 102 and the base portion 104 is not reduced. As a result, the force required for moving the optical head portion 101 can be reduced, and hence a user can move the optical head portion 101 with a small operation force.

As described above, with the ophthalmic apparatus 100 according to the first embodiment, the operations of positioning can be performed while consistently holding the operation stick 108 by one hand at the time of rough positioning and fine positioning. Moreover, at the time of rough positioning, when the operation stick 108 is tilted, the urging force generated between the base portion 104 and the movable portion 102 is reduced, and hence the force required for moving the movable portion 102 can be reduced. Accordingly, with the ophthalmic apparatus 100 according to the first embodiment, the rough positioning can be performed without degradation in operational feeling.

Moreover, the urging force reduction mechanism of the first embodiment has the angle regulation surface 403 formed on the movable portion 102, and when the operation stick 108 is tilted by the predetermined angle or larger, the angle regulation surface 403 is brought into abutment against the operation stick 108 to support the operation stick 108. When the operation stick 108 is supported by the angle regulation surface 403, the friction portion 223 is brought into non-contact with the friction plate 222, and hence the urging force of the friction portion 223 against the friction plate 222 is reduced. With this, the friction force generated between the friction portion 223 and the friction plate 222 is reduced. Therefore, the force required for moving the optical head portion 101 can be further reduced, and hence a user can move the optical head portion 101 with a smaller operation force.

### Second Embodiment

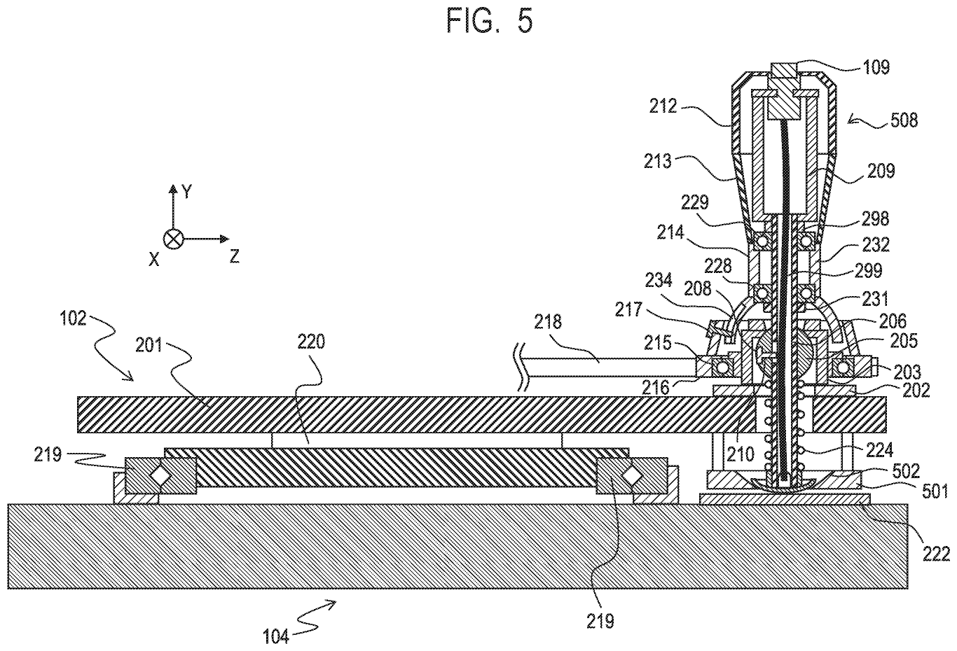

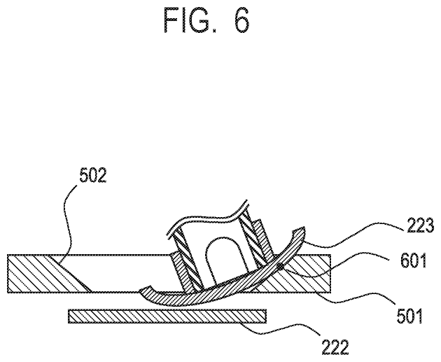

In a second embodiment, a configuration different from the configuration of the first embodiment is used to reduce the urging force generated between the base portion 104 and the movable portion 102 at the time of rough positioning. With regard to the configuration of the second embodiment, configurations which are common to the first embodiment are denoted by the same reference symbols, and description thereof is omitted. In the following, with reference to FIG. 5 and FIG. 6, the ophthalmic apparatus 100 according to the second embodiment is described mainly on differences from the first embodiment.

FIG. 5 is a view for schematically illustrating an example of a cross-sectional configuration of an internal structure of the movable portion 102, the base portion 104, and an operation stick 508 of the ophthalmic apparatus 100, and is a sectional view taken along a plane extending along the center of the imaging button 109 of the operation stick 508 and being orthogonal to the X axis. The configuration of the first embodiment illustrated in FIG. 2A and FIG. 2B and the configuration of the second embodiment illustrated in FIG. 5 are different from each other in the presence or absence of the annular member 238 and a friction-portion receiving member 501.

The annular member 238 is not provided to the operation stick 508 of the second embodiment. Meanwhile, the friction-portion receiving member 501 is fixed by a screw (not shown) to a lower surface of the movable-portion base 201. The friction-portion receiving member 501 has an inclined surface 502 having an axisymmetric shape. The friction-portion receiving member 501 is provided so that the axis of the center shaft 206 substantially matches a symmetry axis of the inclined surface 502.

<Operation of Rough Positioning>

Here, an operation of rough positioning of the optical head portion 101 in the X-Z direction (horizontal direction) in the second embodiment is described. FIG. 6 schematically shows a positional relationship between the friction portion 223 and the friction-portion receiving member 501 at the time of tilting the operation stick 508. In FIG. 6, for simple description, the electric harness 299 is omitted. An operation of fine positioning in the second embodiment is the same as the operation of the fine positioning in the first embodiment, and hence description is omitted.

In the case of the rough positioning of the optical head portion 101, under a state in which the exterior cover 212 of the operation stick 508 is held by a user such as an inspector, the operation stick 508 is tilted in a direction of moving the optical head portion 101. When the operation stick 508 is tilted, as illustrated in FIG. 6, a spherical-surface portion of the friction portion 223 is in contact with a contact point 601 of the inclined surface 502 of the friction-portion receiving member 501. At the contact point 601, the friction portion 223 receives a reaction force (not shown) from the friction-portion receiving member 501.

When the friction portion 223 receives the reaction force from the friction-portion receiving member 501, the urging force generated between the friction portion 223 and the friction plate 222 is reduced, and hence the friction force generated between the friction portion 223 and the friction plate 222 is reduced. Therefore, the friction-portion receiving member 501 functions as an example of the urging force reduction mechanism arranged to reduce the urging force generated between the friction portion 223 and the friction plate 222 to reduce the urging force generated between the base portion 104 and the movable portion 102.

Here, when the operation stick 508 is tilted by a predetermined angle or larger, the urging force reduction mechanism is capable of reducing the urging force generated between the base portion 104 and the movable portion 102 based on the reaction force at the movable portion 102 against the operation force applied to the operation stick 508. More specifically, the urging force reduction mechanism is capable of reducing the urging force between the base portion 104 and the movable portion 102 through use of the force in the axial direction of the operation stick 508 generated by the reaction force.

When the friction force generated between the friction portion 223 and the friction plate 222 is reduced by reducing the urging force generated between the base portion 104 and the movable portion 102, the operation force for the rough positioning is reduced as compared to a state in which the urging force generated between the friction portion 223 and the friction plate 222 is not reduced. As a result, a force required for moving the optical head portion 101 can be reduced, and hence a user can move the optical head portion 101 with a small operation force.

Here, when the operation stick 508 is tilted to an angle larger than the predetermined angle, while the friction portion 223 slides on the inclined surface 502 of the friction portion receiving member 501, the operation stick 508 tilts about the inner ball 205 as a center. At this time, the friction portion 223 rides over the inclined surface 502 while the friction portion 223 slides on the inclined surface 502 of the friction portion receiving member 501, thereby allowing the center shaft 206 of the operation stick 508 to move in a direction of separating away from the friction plate 222 in the axial direction relative to the inner ball 205 accommodated in the inner-ball base 203 of the movable portion 102.

More specifically, when the force in the axial direction of the operation stick 508 generated by the moment of the operation force and the reaction force received from the inclined surface 502 becomes larger than the urging force of the coil spring 224 (urging member) at the time of fine positioning, the operation stick 508 moves in the direction in which the length of the coil spring 224 is shortened in the axial direction. In the operation stick 508, at the time of fine positioning, the urging force of the coil spring 224 between the inner ball 205 and the friction portion 223 causes the friction portion 223 to be urged toward the friction plate 222, thereby bringing the friction portion 223 and the friction plate 222 into contact with each other. In contrast, when the operation stick 508 moves in the direction in which the length of the coil spring 224 becomes shorter than the length of the coil spring 224 at the time of fine positioning, the operation stick 508 is brought into a state in which the friction portion 223 is separated away from the friction plate 222.

In other words, when the operation stick 508 is tilted by the predetermined angle or larger, the shaft portion (center shaft 206) of the operation stick 508 moves in the axial direction relative to the movable portion 102 so that a distance between the lower portion (friction portion 223) of the operation stick 508 and the movable portion 102 in the axial direction of the operation stick 508 becomes shorter than the distance given when a tilt angle of the operation stick 508 falls within the range of from 0 degrees to the predetermined angle. With this, the urging force reduction mechanism is capable of reducing the urging force between the base portion 104 and the movable portion 102.

When the operation stick 508 moves in the direction in which the length of the coil spring 224 becomes shorter than the length of the coil spring 224 at the time of fine positioning, the urging force of the coil spring 224 also becomes larger. In contrast, with the configuration described above, a distance between a point of action to which the operation force is applied and the inner ball 205 can be set sufficiently longer than a distance between the point of action to which the urging force is applied and the inner ball 205. Moreover, when the friction portion 223 rides over the inclined surface 502, the operation stick 508 receives the force in the direction in which the friction portion 223 moves away from the friction plate 222 in the axial direction by the reaction force received from the inclined surface 502. Therefore, the operation force required for achieving the moment of the operation force larger than the moment of the urging force can be set to a small force. Thus, a user can reduce the urging force generated between the base portion 104 and the movable portion 102 with the operation force smaller than the force for moving the operation stick 508 in the axial direction, and can perform the rough positioning.

Moreover, when a user keeps applying the operation force to the operation stick 508 so that the moment of the operation force with the inner ball 205 as a rotation center is balanced with the moment of the urging force, the state in which the urging force is reduced can be maintained. Therefore, the state in which the force required for moving the optical head portion 101 at the time of rough positioning is reduced can be maintained. Here, with the configuration described above, a distance between the point of action to which the operation force is applied and the inner ball 205 can be set sufficiently longer than a distance between the point of action to which the urging force is applied and the inner ball 205. Moreover, when the friction portion 223 rides over the inclined surface 502, the operation stick 508 receives the force in the direction in which the friction portion 223 separates away from the friction plate 222 in the axial direction by the reaction force received from the inclined surface 502. Therefore, the operation force required for achieving the moment of the operation force balanced with the moment of the urging force can be set to a small force. Thus, a user can perform the rough positioning with a smaller operation force.

As described above, the urging force reduction mechanism of the second embodiment includes the friction-portion receiving member 501 provided to the movable portion 102. The friction portion 223 is brought into contact with the friction-portion receiving member 501 when the operation stick 508 is tilted by a predetermined angle or larger. When the friction portion 223 is brought into contact with the friction-portion receiving member 501, the reaction force against the operation force applied to the operation stick 508 is applied from the movable portion 102 to the operation stick 508. Based on the reaction force, the urging force of the friction portion 223 against the friction plate 222 is reduced.

In particular, the friction-portion receiving member 501 includes the inclined surface 502 having a symmetry axis which is substantially equivalent to a center-shaft symmetry axis of the operation stick 508 when the operation stick 508 is not tilted. Moreover, the friction portion 223 is brought into contact with the inclined surface 502 when the operation stick 508 is tilted by the predetermined angle or larger.

With this, the urging force generated between the movable portion 102 and the base portion 104 is reduced, and hence the friction force generated between the friction portion 223 and the friction plate 222 is reduced. Therefore, the operation force for the rough positioning is reduced as compared to the state in which the urging force generated between the movable portion 102 and the base portion 104 is not reduced. As a result, the force required for moving the optical head portion 101 can be reduced, and hence a user can move the optical head portion 101 with a small operation force.

As described above, also with the ophthalmic apparatus 100 according to the second embodiment, the operations of positioning can be performed while consistently holding the operation stick 508 by one hand at the time of rough positioning and fine positioning. Moreover, at the time of rough positioning, when the operation stick 508 is tilted, the urging force generated between the base portion 104 and the movable portion 102 is reduced, and hence the force required for moving the movable portion 102 can be reduced. Accordingly, also with the ophthalmic apparatus 100 according to the second embodiment, the rough positioning can be performed without degradation in operational feeling.

The friction portion 223 may be brought into non-contact with the friction plate 222 when the friction portion 223 is brought into contact with the friction-portion receiving member 501. In this case, the friction-portion receiving member 501 may function as a supporting portion which is arranged to be brought into abutment against the operation stick 508 to support the operation stick 508 when the operation stick 508 is tilted by the predetermined angle or larger. When the operation stick 508 is supported by the friction-portion receiving member 501, the friction portion 223 is brought into non-contact with the friction plate 222, and hence the urging force of the friction portion 223 against the friction plate 222 is reduced. Therefore, the force required for moving the movable portion 102 can be further reduced, and hence a user can move the optical head portion 101 with a smaller operation force.

### Third Embodiment

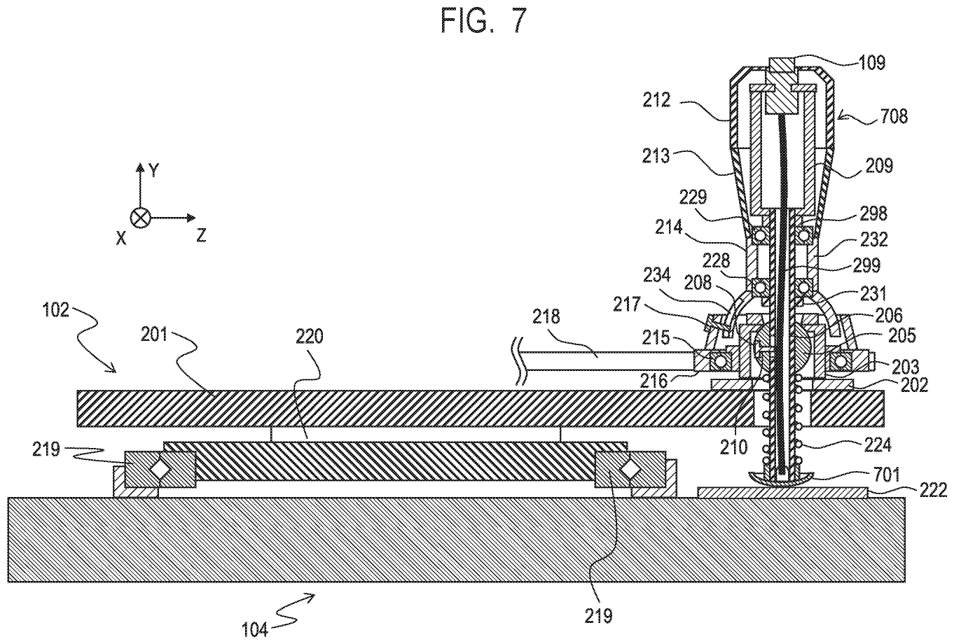

In a third embodiment, a configuration different from the configurations of the first and second embodiments is used to reduce the urging force generated between the base portion 104 and the movable portion 102 at the time of rough positioning. Configurations which are common to the first and second embodiments are denoted by the same reference symbols, and description thereof is omitted. In the following, with reference to FIG. 7 to FIG. 9, the ophthalmic apparatus 100 according to the third embodiment is described mainly on differences from the first embodiment.

FIG. 7 is a view for schematically illustrating an example of a cross-sectional configuration of an internal structure of the movable portion 102, the base portion 104, and an operation stick 708 of the ophthalmic apparatus 100, and is a sectional view taken along a plane extending along the center of the imaging button 109 of the operation stick 708 and being orthogonal to the X axis. The configuration of the first embodiment illustrated in FIG. 2A and FIG. 2B and the configuration of the third embodiment illustrated in FIG. 7 are different from each other in the presence or absence of the annular member 238 and in the configuration of the friction portion 223 and a friction portion 701.

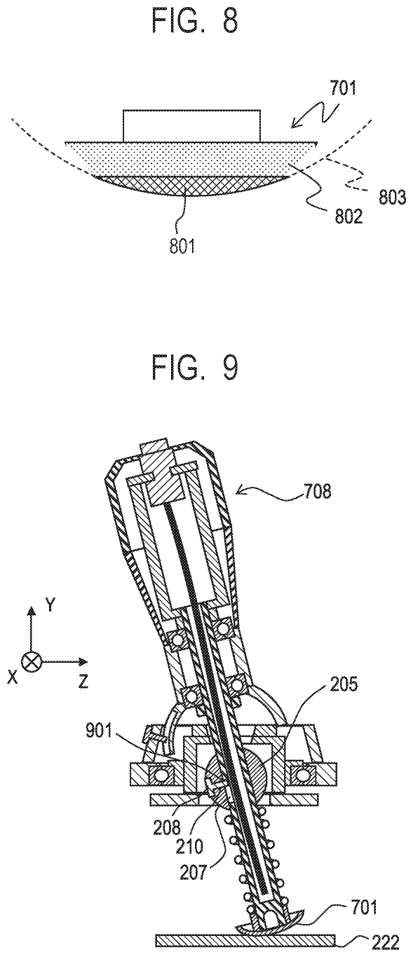

FIG. 8 is a view for illustrating a shape of the friction portion 701 provided to the operation stick 708 of the third embodiment. Similarly to the friction portion 223 of the first and second embodiments, the friction portion 701 is fixed by a screw (not shown) to the lower end portion of the center shaft 206 of the operation stick 708. With regard to the friction portion 223 of the first and second embodiments, the spherical-surface portion which rolls on the surface of the friction plate 222 has a shape having a uniform spherical surface radius. Meanwhile, the friction portion 701 includes, as illustrated in FIG. 8, a spherical-surface portion 801 having a first spherical surface radius (first curvature radius) and a spherical-surface portion 802 (outer-surface portion) having a second spherical surface radius (second curvature radius).

The first spherical surface radius is substantially equal to a distance from the center of the inner ball 205 to the upper surface of the friction plate 222, and a movement resolution of the fine positioning (movement amount of the optical head portion 101 in accordance with a tilt angle of the operation stick 708) is determined based on the first spherical surface radius. The second spherical surface radius is smaller than the first spherical surface radius. However, it is not always required that the spherical-surface portion 802 be a spherical surface. It is only required that the portion 802 have a shape which is recessed more than a spherical surface 803 having the spherical surface radius of the spherical-surface portion 801 as indicated by the broken line in FIG. 8, in other words, a shape of approaching the inner ball 205 side.

<Operation of Rough Positioning>

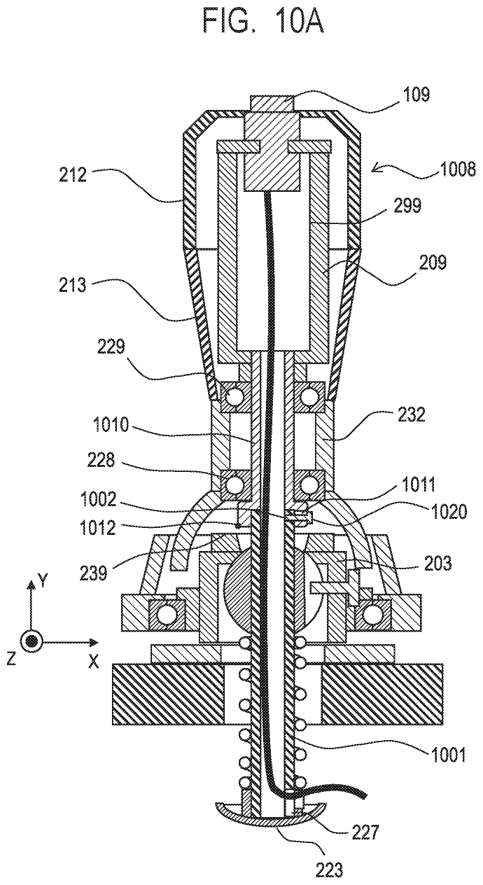

Here, an operation of rough positioning of the optical head portion 101 in the X-Z direction (horizontal direction) according to the third embodiment is described. FIG. 9 is a view for illustrating a state in which the operation stick 708 is tilted by a user at the time of rough positioning. An operation of fine positioning in the third embodiment is the same as the operation of the fine positioning in the first embodiment, and hence description is omitted.

In the case of the rough positioning of the optical head portion 101, the operation stick 708 is tilted by a user such as an inspector. When the operation stick 708 is tilted, as the spherical-surface portion 802 of the friction portion 701 has a shape which is recessed more than the spherical-surface portion 801, the operation stick 708 moves toward the −Y direction side in the axial direction, and the friction portion 701 moves away from the movable portion 102. At this time, an upper abutment portion 901 of the elongated hole 207 of the center shaft 206 is brought into abutment against the regulation pin 208. With this, a distance by which the friction portion 701 and the inner ball 205 are separated away from each other by the coil spring 224 is regulated, and hence the inner ball 205 is prevented from being urged by the coil spring 224 against the inner peripheral surface of the inner-ball base 203. Therefore, the friction force generated between the inner ball 205 and the inner peripheral surface of the inner-ball base 203 is reduced. As illustrated in FIG. 9, when the elongated hole 207 is brought into abutment against the regulation pin 208 to restrict the movement of the inner ball 205, the inner ball 205 may be brought into non-contact with the inner peripheral surface of the inner-ball base 203.

Similarly, when the elongated hole 207 is brought into abutment against the regulation pin 208 to restrict the movement of the inner ball 205, the friction portion 701 (in particular, portion such as the spherical-surface portion 802, which is a lower surface of the friction portion 701 and is formed into the spherical surface shape) is prevented from being urged by the coil spring 224 against the friction plate 222. As a result, the friction force generated between the friction portion 701 and the friction plate 222 is reduced. Therefore, the spherical-surface portion 802 functions as an example of an urging force reduction mechanism arranged to reduce the urging force generated between the friction portion 701 and the friction plate 222 to reduce the urging force generated between the base portion 104 and the movable portion 102.

As described above, the urging force reduction mechanism of the third embodiment includes the spherical-surface portion 802 (outer-surface portion), which has a curvature radius smaller than a curvature radius of the spherical-surface portion 801 formed on the friction portion 701 and is formed on a peripheral edge portion of the spherical-surface portion 801. When the operation stick 708 is tilted by the predetermined angle or larger, and the spherical-surface portion 802 is brought into abutment against the friction plate 222, the friction portion 701 moves away from the movable portion 102, and hence the urging force generated by the urging portion between the base portion 104 and the movable portion 102 is reduced.

More specifically, the urging portion includes the coil spring 224 arranged to urge the inner ball 205 and the friction portion 701, which are provided to the operation stick 708, in a longitudinal direction of the operation stick 708, and the movable portion 102 includes the inner-ball base 203 arranged to accommodate the inner ball 205. Moreover, the curvature radius of the spherical-surface portion 801 of the friction portion 701 is substantially equal to the distance from the center of the inner ball 205 to the friction plate 222.

Further, the operation stick 708 includes the regulation pin 208 (regulation member) arranged to regulate the movement of the operation stick 708 in the longitudinal direction relative to the inner ball 205. When the operation stick 708 is tilted by the predetermined angle or larger, the regulation pin 208 regulates the movement of the operation stick 708 in the longitudinal direction relative to the inner ball 205. With this, a distance by which the friction portion 701 and the inner ball 205 are separated away from each other by the coil spring 224 is regulated. Thus, in accordance with the tilt or movement of the operation stick 708, the inner ball 205 separates from the inner-ball base 203, and the friction portion 701 separates from the movable portion 102. Therefore, the urging force generated by the urging portion between the base portion 104 and the movable portion 102 is reduced. In particular, the regulation pin 208 is inserted into the screw hole 210 (hole portion), which is formed in the inner ball 205, and the elongated hole 207 (hole portion), which is formed in the operation stick 708 and extends in the longitudinal direction of the operation stick 708.

As a result, the urging force generated between the inner ball 205 and the friction portion 701 is reduced, and hence the friction force generated between the inner ball 205 and the inner peripheral surface of the inner-ball base 203 of the movable-portion base 201 and the friction force generated between the friction portion 701 and the friction plate 222 of the base portion 104 are reduced. With this, the urging force generated between the base portion 104 and the movable portion 102 is reduced, and hence the friction force generated between the friction portion 701 and the friction plate 222 is reduced. Therefore, the force required for moving the optical head portion 101 can be reduced, and hence a user can move the optical head portion 101 with a small operation force.

As described above, also with the ophthalmic apparatus 100 according to the third embodiment, the operations of positioning can be performed while consistently holding the operation stick 708 by one hand at the time of rough positioning and fine positioning. Moreover, at the time of rough positioning, when the operation stick 708 is tilted, the urging force generated between the base portion 104 and the movable portion 102 is reduced, and hence the force required for moving the movable portion 102 can be reduced. Accordingly, also with the ophthalmic apparatus 100 according to the third embodiment, the rough positioning can be performed without degradation in operational feeling.

### First Modification Example

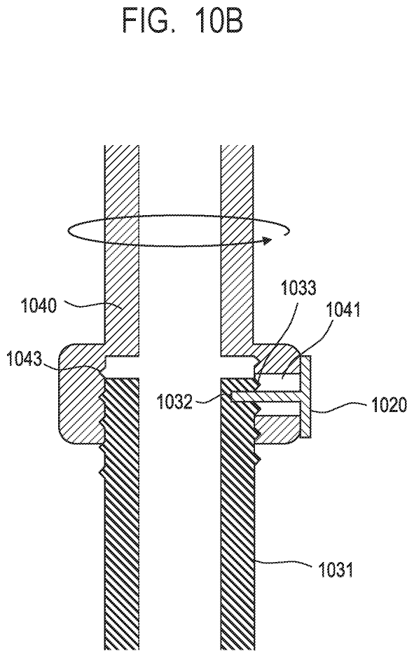

In the first embodiment, the annular member 238 is fixed to the center shaft 206 by a screw (not shown) and is provided below the protruding portion 231 and above the inner ball 205. Here, when the annular member 238 and the center shaft 206 are separate members, in order to change a movable range of the fine positioning, the annular member 238 may have a structure with which a position of mounting to the operation stick 108 can be adjusted in the axial direction of the center shaft 206 of the operation stick 108. With this, the predetermined angle as the tilt angle of the operation stick 108 can be adjusted. Now, with reference to FIG. 10A, an example of an ophthalmic apparatus having such a structure is described.

FIG. 10A is a view for schematically illustrating an example of a cross-sectional configuration of an internal structure of the movable portion 102, the base portion 104, and an operation stick 1008 of the ophthalmic apparatus 100, and is a sectional view taken along a plane extending along the center of the imaging button 109 of the operation stick 1008 and being orthogonal to the Z axis. The operation stick 1008 of the first modification example includes a center shaft 1001 in place of the center shaft 206, and an annular member 1010 in place of the annular member 238. Other configurations are the same as the configurations of the first embodiment, and hence description is omitted.

The center shaft 1001, similarly to the center shaft 206, is a hollow shaft. The center shaft 1001 is inserted through the through hole formed in the inner ball 205, and is reciprocally movable in the axial direction of the center shaft 206. Moreover, similarly to the center shaft 206, the center shaft 1001 has the elongated hole 207 and the cutout portion 227, and the friction portion 223 is fixed to a lower end portion of the center shaft 1001 by a screw (not shown). Moreover, a screw hole 1002, which is a through hole allowing communication between an inner periphery side and an outer periphery side of the center shaft 1001, is formed at a top portion of the center shaft 1001. The screw hall 1002 may not be a through hole.

The annular member 1010 is a hollow shaft extending coaxially with the center shaft 1001 in the axial direction. The relay member 209 is fixed to a top portion of the annular member 1010 by a screw (not shown). Moreover, the exterior covers 212 to 214 of the operation stick 708 are arranged so as to cover the annular member 1010 and the relay member 209 and are rotatable coaxially with the annular member 1010 and the center shaft 1001 of the operation stick 708. Here, configurations of the exterior covers 212 to 214 and the deep groove ball bearings 228 and 229 may be the same as the configurations of the first embodiment, but the inner rings of the deep groove ball bearings 228 and 229 are arranged to fit the annular member 1010.

At the lower portion of the annular member 1010, the annular member 1010 is formed so that an inner peripheral surface of the annular member 1010 faces an outer peripheral surface of the center shaft 1001 to be capable of covering (accommodating) the center shaft 1001. Moreover, at the portion of the annular member 1010 covering the center shaft 1001, an elongated hole 1011 extending in the longitudinal direction (axial direction) of the annular member 1010 is formed. The elongated hole 1011 is a through hole which allows communication between an inner periphery side and an outer periphery side of the annular member 1010.

Here, the annular member 1010 is arranged such that a position of mounting to the operation stick 1008 can be adjusted in the axial direction of the operation stick 1008. Specifically, the annular member 1010 covers the center shaft 1001 so that positions of the screw hole 1002 and the elongated hole 1011 in the circumferential direction match each other, and the annular member 1010 and the center shaft 1001 are fastened to each other by a screw 1020 passing through the screw hole 1002 and the elongated hole 1011. Here, positions in the longitudinal direction of the elongated hole 1011 to be fastened to the screw hole 1002, that is, positions of the annular member 1010 and the center shaft 1001 in the axial direction are changed through use of the screw 1020, thereby being capable of adjusting the position of the annular member 1010 in the axial direction of the center shaft 1001 of the operation stick 1008.

With such a configuration, the position of a bottom portion (corner portion) of the annular member 1010 with respect to the inner-ball base 203 in the axial direction can be changed by changing the position of the elongated hole 1011 to be fastened to the screw hole 1002 in the longitudinal direction. Therefore, when the operation stick 1008 is tilted in the operation of rough positioning, a position of the corner portion 1012 of the annular member 1010 in the axial direction to be brought into abutment against the upper surface 239 of the inner-ball base 203 can be adjusted, thereby being capable of changing the movable range of the fine positioning. The adjustment of the annular member 1010 may be performed, for example, in a factory at the time of shipment or assembly of the ophthalmic apparatus. Moreover, the elongated hole 1011 of the annular member 1010 and the screw hole 1002 of the center shaft 1001 are not limited to the configuration in which one elongated hole 1011 and one screw hole 1002 are formed. For example, a plurality of elongated holes 1011 may be formed on a circumference of the annular member 1010. With this, finer positional adjustment can be performed. Moreover, a plurality of screw holes 1002 may be formed on a circumference of the center shaft 1001. Moreover, a plurality of elongated holes 1011 and a plurality of screw holes 1002 may be formed. For example, three elongated holes 1011 may be formed at 120-degree intervals, and three screw holes 1002 may be formed at 120-degree intervals. In such a case, the annular member 1010 and the center shaft 1001 can be fastened to each other through use of a plurality of screws 1020.

In this modification example, a position at which the screw hole 1002 and the elongated hole 1011 are fastened to each other by the screw 1020 is changed to change a position of the bottom portion (corner portion) of the annular member 1010 in the axial direction with respect to the inner-ball base 203. Meanwhile, as illustrated in FIG. 10B, there may be formed a screw thread 1033 at a distal end portion of the center shaft 1031 and a thread groove 1043 corresponding to the screw thread 1033 in an inner wall at a lower portion of the annular member 1040, and the center shaft 1031 and the annular member 1040 may be threadedly engaged with each other. The center shaft 1031 and the annular member 1040 are members having functions similar to those of the center shaft 1001 and the annular member 1010, respectively. The screw thread 1033 and the thread groove 1043 construct the feed screw mechanism. In this case, through rotation of the annular member 1040 or the center shaft 1031, a position of the center shaft 1031 in the longitudinal direction with respect to the annular member 1040 can be changed, and a position of the bottom portion (corner portion) of the annular member 1040 in the axial direction with respect to the inner-ball base 203 can be changed. The thread groove may be formed at the distal end portion of the center shaft 1031, and the screw thread corresponding to the thread groove may be formed in the inner wall at the lower portion of the annular member 1040.

Moreover, an elongated hole 1041 extending in the longitudinal direction (axial direction) may be formed in the annular member 1040, and a screw hole 1032 may be formed in the center shaft 1031. In this case, a position of the center shaft 1031 in the longitudinal direction with respect to the annular member 1040 can be fixed by fastening the screw hole 1032 and the elongated hole 1041 with the screw 1020. The elongated hole 1041 is a through hole which allows an inner peripheral side and an outer peripheral side of the annular member 1040 to communicate with each other. Meanwhile, the screw hole 1032 may be a through hole which allows an inner peripheral side and an outer peripheral side of the center shaft 1031 to communicate with each other, or may be other than a through hole. Moreover, also in this case, a plurality of elongated holes 1041 and a plurality of screw holes 1032 may be formed. In such a case, the annular member 1040 and the center shaft 1031 can be fastened to each other through use of a plurality of screws 1020. Moreover, only one of the elongated hole 1041 and the screw hole 1032 may be formed at a plurality of positions in the circumferential direction. In such a case, a position of the center shaft 1031 in the longitudinal direction with respect to the annular member 1040 can be more finely adjusted, and fixation can be performed.

### Fourth Embodiment

<Configuration of Ophthalmic Apparatus>

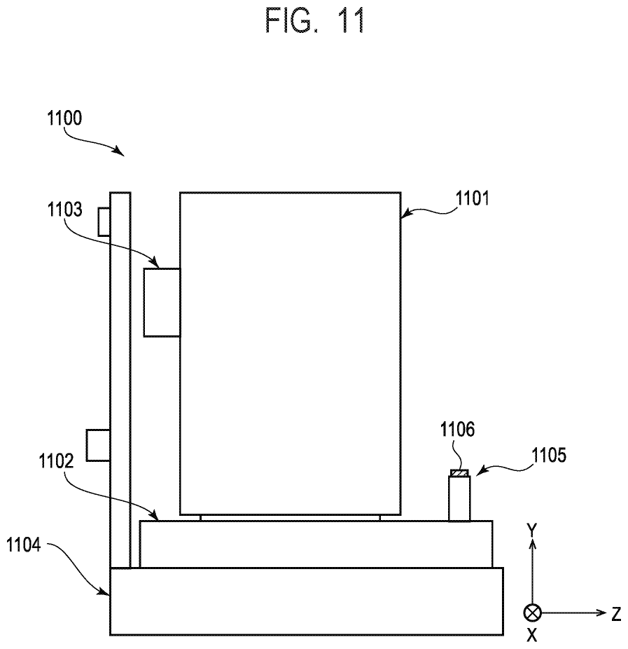

FIG. 11 is a view for illustrating an example of a configuration of an ophthalmic apparatus 1100 according to a fourth embodiment. The ophthalmic apparatus 1100 includes, for example, an optical head portion 1101, a movable portion 1102, a base portion 1104, and an operation stick 1105.

The optical head portion 1101 includes various optical systems for measuring an eye to be inspected. The optical head portion 1101 includes, for example, an object lens portion 1103, an OCT unit, an SLO unit, an anterior-eye observation unit, and a fixation lamp unit.

The object lens portion 1103 is arranged to emit laser to an eye to be inspected and obtain scattered light having returned from the eye to be inspected. The OCT unit and the SLO unit are arranged to obtain a tomographic image and a fundus image of an eye to be inspected based on scattered light information of laser emitted from the object lens portion 1103, respectively. The OCT unit includes an optical system adapted to optical coherence tomography (OCT). The SLO unit includes an optical system adapted to a scanning laser ophthalmoscope (SLO). The anterior-eye observation unit is arranged to image an anterior eye portion of an eye to be inspected and display an anterior eye portion image on a display portion such as a monitor. An inspector sees the anterior eye portion image displayed on the display portion and positions the optical head portion 1101 with respect to the eye to be inspected. The fixation lamp unit displays indicators to lead a fixation direction of the eye to be inspected.

The optical head portion 1101 is only required to have a configuration in which an optical system for measuring an eye to be inspected is provided, and a specific configuration of the optical head portion 1101 is not limited. A configuration having hitherto been publicly known may be applied to the optical head portion 1101. Moreover, a configuration of the optical system provided to the optical head portion 1101 and items to be measured are also not particularly limited.

The movable portion 1102 is provided on the base portion 1104 to support the optical head portion 1101 so that the optical head portion 1101 can be raised and lowered in the Y axis direction. Moreover, the movable portion 1102 is movable in an X-Z plane relative to the base portion 1104, which is a horizontal direction, through intermediation of a horizontal movement mechanism described later. When the movable portion 1102 moves in the horizontal direction relative to the base portion 1104, the optical head portion 1101 also moves in the horizontal direction relative to the base portion 1104 integrally with the movable portion 1102. In order to enable the movable portion 1102 to move in the horizontal direction relative to the base portion 1104, the movable portion 1102 and the base portion 1104 are retained in a state of having a clearance in the Y axis direction.

Moreover, the ophthalmic apparatus 1100 includes the raising and lowering mechanism arranged to raise and lower the optical head portion 1101 in the Y axis direction. For example, a feed screw mechanism is applied to the raising and lowering mechanism. A screw shaft is mounted to the movable portion 1102 so that the screw shaft is rotatable about an axis directed along the Y axis direction. Meanwhile, a nut which is threadedly engaged with the screw shaft is mounted to the optical head portion 1101. When the screw shaft rotates, the optical head portion 1101 is raised or lowered in the Y axis direction relative to the movable portion 1102 together with the nut. A motor arranged to rotate the screw shaft is provided to the movable portion 1102. An output shaft of the motor has an axis being orthogonal to the Y axis direction, and rotation of the motor is transmitted to the screw shaft through worm gears provided to the motor and the screw shaft. It is only required that the raising and lowering mechanism be a mechanism capable of raising and lowering the optical head portion 1101 in the Y axis direction, and a specific configuration of the raising and lowering mechanism is not limited. A configuration having hitherto been publicly known may be applied to the raising and lowering mechanism.

An operation stick 1105 and an operation panel are provided to the movable portion 1102. The operation stick 1105 is provided so as to be tiltable in a freely selected direction by 360 degrees within the X-Z plane relative to the movable portion 1102. An imaging button 1106, which is an electrical switch, is provided at an upper end portion of the operation stick 1105. A controller (information processing device) connected to the base portion 1104 starts measuring (for example, imaging) an eye to be inspected through use of the optical head portion 1101 when an operation of the imaging button 1106 is detected.

Moreover, a lock button is provided to the movable portion 1102. The lock button is an operation member to be used for operating a locking mechanism arranged to lock the movement of the movable portion 1102 in the horizontal direction. The locking mechanism is arranged to fix the movable portion 1102 so that the movable portion 1102 cannot move relative to the base portion 1104. When the lock button is pressed, the movable portion 1102 is locked on the base portion 1104 by the locking mechanism so that the movable portion 1102 cannot move in the horizontal direction relative to the base portion 1104. When the lock button is pressed again in this state, the locking of the movable portion 1102 is released so that the movable portion 1102 is movable in the horizontal direction relative to the base portion 1104. As described above, the lock button is a so-called knock-type mechanism. A configuration having hitherto been publicly known may be applied to the locking mechanism for locking the movable portion 1102 on the base portion 1104. The base portion 1104 is grounded to an installation surface of the ophthalmic apparatus 1100. The base portion 1104 supports the movable portion 1102 so that the movable portion 1102 is movable in the horizontal direction.

<Horizontal Movement Mechanism>

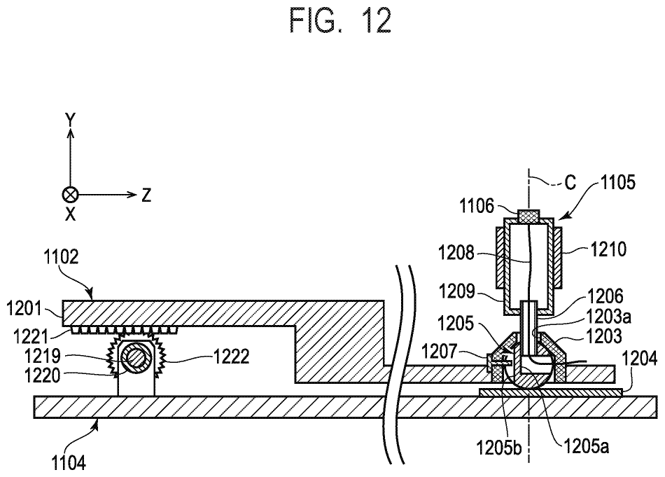

Next, the horizontal movement mechanism arranged to move the movable portion 1102 in the horizontal direction is described. FIG. 12 is a view for illustrating an example of configurations of the movable portion 1102, the base portion 1104, and the operation stick 1105, and is a sectional view taken along a center axis C of the operation stick 1105.

The ophthalmic apparatus 1100 includes the horizontal movement mechanism arranged to move the movable portion 1102 in the horizontal direction. For example, a guide shaft 1219, a rotary slide bush 1220, rack gears 1221, and pinion gears 1222 are applied to the ophthalmic apparatus 1100 as parts of the horizontal movement mechanism. The guide shaft 1219 and the rotary slide bush 1220 are arranged to move the movable portion 1102 in the X axis direction. The rack gears 1221 and the pinion gears 1222 are arranged to move the movable portion 1102 in the Z axis direction.

The rotary slide bush 1220 is retained on the base portion 1104 so that an axis of the rotary slide bush 1220 extends along the X axis direction. The guide shaft 1219 is fitted to the rotary slide bush 1220 so as to be linearly movable in the X axis direction and rotatable about the X axis. The pinion gears 1222 are fixed at both ends of the guide shaft 1219, respectively, and mesh with the two rack gears 1221 provided to the movable portion 1102 so as to extend along the Z axis direction.

Moreover, as components for achieving the horizontal movement function, for example, a friction plate 1204 provided on the base portion 1104 and an inner ball 1205 provided at a lower end portion of the operation stick 1105 are applied. The inner ball 1205 is in contact with an upper surface of the friction plate 1204. When the movable portion 1102 moves in the horizontal direction relative to the base portion 1104, the inner ball 1205 rolls or slides relative to the friction plate 1204. The inner ball 1205 supports part of the weight of the optical head portion 1101 and the movable portion 1102.

With the movement of the movable portion 1102 in the horizontal direction by the horizontal movement mechanism as described above, the optical head portion 1101 can be positioned in the horizontal direction with respect to an eye to be inspected. It is only required that the movable portion 1102 be movable in the horizontal direction relative to the base portion 1104 and that all or part of the weight of the movable portion 1102 be supported by the inner ball 1205, and a specific configuration of the horizontal movement mechanism is not limited. For example, a well-known two-dimensional linear movement stage may be applied to the horizontal movement mechanism.

<Structure of Operation Stick>

The operation stick 1105 includes, for example, the inner ball 1205, a coupling shaft 1206, a stick member 1209, and an exterior cover 1210.

The inner ball 1205 has a spherical shape, and is provided at the lower end portion of the operation stick 1105. The inner ball 1205 corresponds to an example of the friction portion. The inner ball 1205 is accommodated in an inner-ball base 1203 which is mounted to a base part 1201 of the movable portion 1102 and constructs a part of the movable portion 1102. The inner-ball base 1203 is a hollow member and is capable of accommodating the inner ball 1205. Moreover, the inner-ball base 1203 has a through hole 1203a formed at a top thereof. The coupling shaft 1206 of the operation stick 1105 passes through the through hole 1203a, and the through hole 1203a is brought into abutment against the coupling shaft 1206 when the operation stick 1105 is tilted by a predetermined angle or larger. The through hole 1203a corresponds to an example of an abutment portion.

Moreover, the inner ball 1205 has a fitting hole 1205a for fixing the coupling shaft 1206 fitted to the fitting hole 1205a from an upper portion of the inner ball 1205. The fitting hole 1205a is a hole communicating with a side surface of the inner ball 1205 and having a substantially L-shape as seen from the X axis direction. An electric harness 1208 can be inserted through the fitting hole 1205a. The electric harness 1208 is wired from the base portion 1104 to the movable portion 1102. Moreover, a slit 1205b is formed in an outer peripheral surface of the inner ball 1205. A rotation stop pin 1207 is fixed to the inner-ball base 1203, and a distal end of the rotation stop pin 1207 is inserted into the slit 1205b of the inner ball 1205. Thus, rotation of the operation stick 1105 relative to the movable portion 1102 is regulated, and only tilting is allowed. Through the regulation of the rotation of the operation stick 1105, damage on the electric harness 1208 can be prevented. When an inspector releases the operation stick 1105, owing to static friction generated between the inner ball 1205 and the inner-ball base 1203, the movable portion 1102 is retained so as to be prevented from being moved by a reaction force of a bundle of the electric harness 1208.

The coupling shaft 1206 is a hollow shaft-like member, and is fixed to the fitting hole 1205a of the inner ball 1205. The electric harness 1208 is inserted through the coupling shaft 1206 and extends inside along the axial direction. The stick member 1209 is a hollow shaft-like member having a diameter larger than a diameter of the coupling shaft 1206 and is coaxially fixed at the top of the coupling shaft 1206. Moreover, the imaging button 1106 is mounted at an upper end portion of the stick member 1209.

The exterior cover 1210 has a substantially tubular shape and is mounted on an outer periphery of the stick member 1209. The exterior cover 1210 is rotatable about an axis of the stick member 1209. On an inner peripheral portion of the exterior cover 1210, a disc-shaped chopper is mounted coaxially with the stick member 1209. Thus, the exterior cover 1210 and the chopper rotate together. A sensor arranged to detect rotation of the chopper is provided to the stick member 1209. An electric harness (not shown) connected to the sensor is connected to an electric board which is provided on the base part 1201 together with the electric harness 1208. Thus, a rotation angle of the exterior cover 1210 can be measured by the sensor. Information of the rotation angle of the exterior cover 1210 measured by the sensor is used at the time of driving the above-mentioned motor arranged to raise and lower the optical head portion 1101. That is, when the exterior cover 1210 of the operation stick 1105 is rotated by an inspector, the optical head portion 1101 is raised or lowered, thereby being capable of positioning the optical head portion 1101 in the Y axis direction with respect to an eye to be inspected. As the raising and lowering mechanism, a publicly known mechanism which is capable of raising and lowering the optical head portion 1101 in the Y axis direction in accordance with a freely selected operation such as a rotation operation on the operation stick 1105 can be applied. Moreover, the driving source is also not limited to an electric power type such as a motor.

<Friction Reduction Mechanism>

The ophthalmic apparatus 1100 includes a friction reduction mechanism arranged to reduce a friction coefficient between the inner ball 1205 and the friction plate 1204 when the operation stick 1105 is tilted by a predetermined angle or larger. The friction reduction mechanism of the fourth embodiment includes a friction reduction portion, which is provided on a peripheral edge portion of the spherical-surface portion formed on the inner ball 1205.

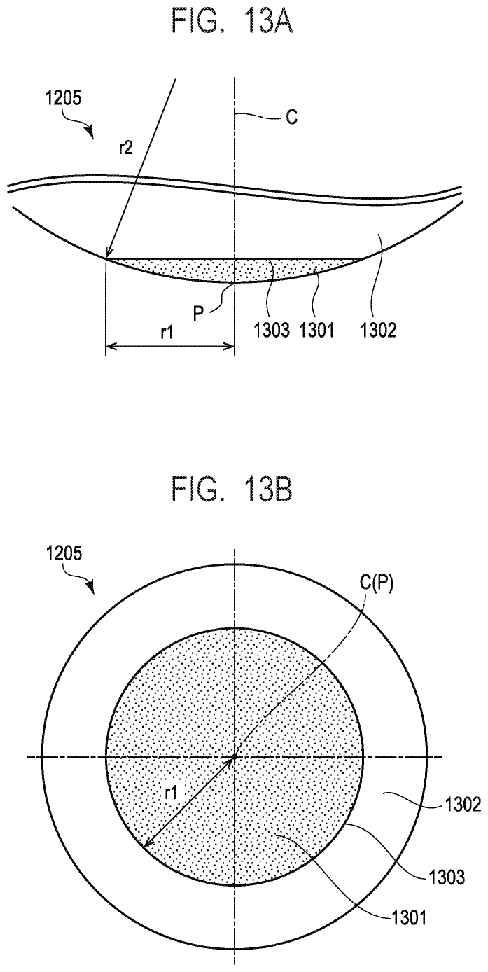

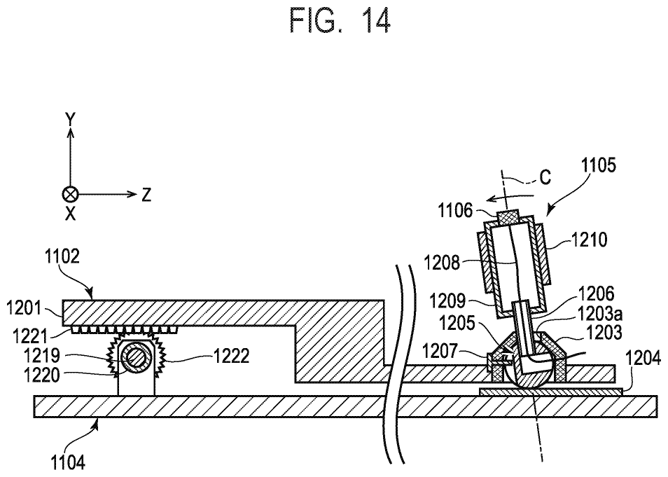

Here, the friction reduction mechanism of the fourth embodiment is described. FIG. 13A is a view for illustrating an example of a configuration of a lower portion of the inner ball 1205. FIG. 13A is a view for illustrating the example of the configuration of the inner ball 1205 as seen from a direction orthogonal to the center axis C of the operation stick 1105, and FIG. 13B is a view for illustrating the example of the configuration of the inner ball 1205 as seen from a lower side along an axis direction of the center axis C.

The inner ball 1205 includes a spherical-surface portion 1301 and an outer-surface portion 1302. The spherical-surface portion 1301 is formed of a surface of the inner ball 1205. As illustrated in FIG. 13A, the spherical-surface portion 1301 is a surface including a lowest end point P of the inner ball 1205 and extending upward from the lowest end point P along the surface of the inner ball 125. Moreover, as illustrated in FIG. 13B, the spherical-surface portion 1301 is a surface having a circular region, which includes a region passing through the center axis C and has a radius r1 with a center at the center axis C.

The outer-surface portion 1302 is formed of the surface of the inner ball 1205 and is provided on a peripheral edge portion of the spherical-surface portion 1301. The spherical-surface portion 1301 and the outer-surface portion 1302 are each a part of the same spherical shape and are provided in different regions on the spherical shape. As illustrated in FIG. 13A, the outer-surface portion 1302 is a surface extending upward from the spherical-surface portion 1301 along the surface of the inner ball 1205. Moreover, as illustrated in FIG. 13B, the outer-surface portion 1302 is an annular surface having a center at the center axis C.

Moreover, as illustrated in FIG. 13A, a boundary line 1303 between the spherical-surface portion 1301 and the outer-surface portion 1302 is orthogonal to the center axis C. Moreover, as illustrated in FIG. 13B, the boundary line 1303 between the spherical-surface portion 1301 and the outer-surface portion 1302 has a circular shape with the radius r1 having a center at the center axis C.

The friction reduction mechanism of the fourth embodiment includes the outer-surface portion 1302. The outer-surface portion 1302 corresponds to an example of a friction reduction portion. The outer-surface portion 1302 has a surface roughness smaller than a surface roughness of the spherical-surface portion 1301. In order to change the surface roughness at a lower portion of the inner ball 1205, for example, when the inner ball 1205 is formed by cutting, the surface roughness can be changed with a difference in feed speed of a cutting tool. Moreover, the surface roughness may be reduced by mechanical polishing such as buffing, or the surface roughness may be changed by electrolytic polishing or chemical polishing.

A tilt angle formed between the Y axis and the center axis C of the operation stick 1105 when the operation stick 1105 is tilted is represented by θ. A radius of the boundary line between the spherical-surface portion 1301 and the outer-surface portion 1302 is represented by r1, and a radius of the inner ball 1205 is represented by r2. The tilt angle θ corresponds to an example of a predetermined angle.

In this case, when the tilt angle θ of the operation stick 1105 is smaller than sin⁻¹(r1/r2), the spherical-surface portion 1301, which has a larger surface roughness on the inner ball 1205, and the friction plate 1204 are brought into contact with each other. Meanwhile, when the tilt angle θ of the operation stick 1105 is equal to or larger than sin⁻¹(r1/r2), the outer-surface portion 1302, which has a smaller surface roughness on the inner ball 1205, and the friction plate 1204 are brought into contact with each other.

<Operations of Fine Positioning and Rough Positioning in Horizontal Direction>

Next, an action of the friction reduction mechanism at the time of moving the optical head portion 1101 in the horizontal direction is described with reference to FIG. 14. FIG. 14 is a view for illustrating a state in which the operation stick 1105 is tilted. First, an operation of fine positioning of the optical head portion 1101 in the horizontal direction is described. When the operation stick 1105 is tilted by an inspector in a moving direction of the optical head portion 1101, the inner ball 1205 rolls on the surface of the friction plate 1204 without slipping. At this time, the spherical-surface portion 1301 of the inner ball 1205 is in contact with the friction plate 1204. A movement resolution of the fine positioning (movement amount of the optical head portion 1101 in accordance with a tilt angle of the operation stick 1105) is determined based on the radius r2 of the inner ball 1205.

Next, an operation of rough positioning of the optical head portion 1101 in the horizontal direction is described. When the operation stick 1105 is further tilted to an angle equal to or larger than the predetermined angle from the fine positioning operation, the coupling shaft 1206 is brought into abutment against an inner peripheral surface of the through hole 1203a formed at the top of the inner-ball base 1203. Further, when a load in the horizontal direction acts on the operation stick 1105, the inner ball 1205 slides relative to the friction plate 1204. At this time, the outer-surface portion 1302 of the inner ball 1205 is in contact with the friction plate 1204, and a friction coefficient on a contact surface between the inner ball 1205 and the friction plate 1204 is smaller than the friction coefficient given in the case in which the spherical-surface portion 1301 of the inner ball 1205 is in contact with the friction plate 1204.

Thus, in the case of the rough positioning, the friction coefficient on the contact surface between the inner ball 1205 and the friction plate 1204 is smaller than that given in the case of the fine positioning. Here, with regard to the load in the horizontal direction required for moving the optical head portion 1101, slide resistance in the horizontal direction generated on the contact surface between the friction plate 1204 and the inner ball 1205 (that is, a product of a perpendicular reaction force on the contact surface with the friction coefficient) is dominant. As described above, as the friction coefficient on the contact surface between the inner ball 1205 and the friction plate 1204 becomes smaller, the slide resistance in the horizontal direction generated on the contact surface between the inner ball 1205 and the friction plate 1204 becomes smaller. As a result, the force required for moving the optical head portion 1101 can be reduced. Moreover, in the case of performing the rough positioning and the fine positioning, an inspector can perform the operations while consistently holding the operation stick 1105 by one hand.

### Fifth Embodiment

Next, an ophthalmic apparatus according to a fifth embodiment is described. A configuration of an inner ball 1501 of the ophthalmic apparatus according to the fifth embodiment is different from the configuration of the inner ball 1205 of the fourth embodiment. Configurations of the inner ball 1501 of the fifth embodiment which are the same as the configurations of the fourth embodiment are denoted by the same reference symbols, and description is omitted.

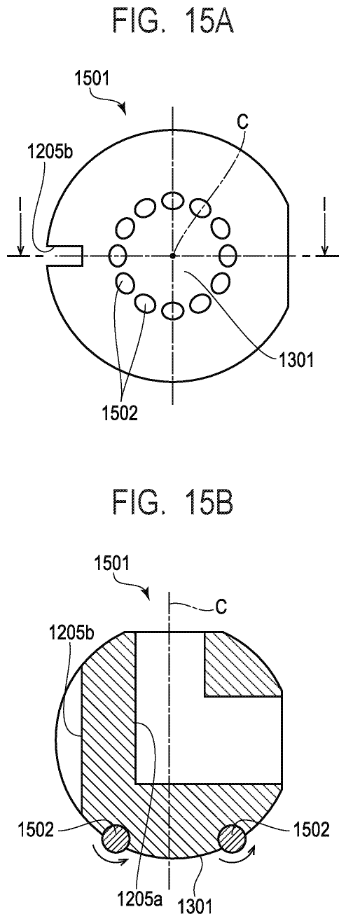

FIG. 15A and FIG. 15B are views for illustrating an example of a configuration of the inner ball 1501 of the fifth embodiment. FIG. 15A is a view for illustrating the example of the configuration of the inner ball 1501 as seen from the lower side along the axis direction of the center axis C. FIG. 15B is a sectional view for illustrating the inner ball 1501 taken along the line I-I illustrated in FIG. 15A. The inner ball 1501 includes the spherical-surface portion 1301 and rolling elements 1502. The spherical-surface portion 1301 has the same configuration as the fourth embodiment.

The rolling elements 1502 are retained on the peripheral edge portion of the spherical-surface portion 1301 so as to be rollable. Specifically, a plurality of rolling elements 1502 are arranged at intervals along the peripheral edge portion of the spherical-surface portion 1301. Balls of a general ball bearing can be applied as the rolling elements 1502. The friction reduction mechanism of the fifth embodiment includes the rolling elements 1502. The rolling elements 1502 correspond to an example of the friction reduction portion.

Next, an action of the friction reduction mechanism at the time of moving the optical head portion 1101 in the horizontal direction is described. The operation of fine positioning of the optical head portion 1101 in the horizontal direction is the same as the fourth embodiment.

Next, an operation of rough positioning of the optical head portion 1101 in the horizontal direction is described. When the operation stick 1105 is further tilted to the predetermined angle or larger from the operation of fine positioning, the coupling shaft 1206 is brought into abutment against the inner peripheral surface of the through hole 1203a formed at the top of the inner-ball base 1203. At this time, the rolling elements 1502 retained on the inner ball 1501 are brought into a state of being in contact with the friction plate 1204. When the load in the horizontal direction further acts on the operation stick 1105, through the movement of the optical head portion 1101 in the horizontal direction, the rolling elements 1502 roll relative to the friction plate 1204.

Thus, in the case of the rough positioning, resistance in the horizontal direction generated between the rolling elements 1502 and the friction plate 1204 is caused by rolling friction, and hence the resistance becomes smaller than the friction caused by slip resistance when the perpendicular reaction force does not change. As described above, in the case of the rough positioning, the resistance in the horizontal direction on the contact surface between the inner ball 1501 and the friction plate 1204 becomes smaller. As a result, the force required for moving the optical head portion 1101 can be reduced.

In the fourth embodiment and the fifth embodiment described above, the ophthalmic apparatus 1100 includes the friction reduction mechanism arranged to reduce the friction coefficient between the inner ball 1205, 1501 and the friction plate 1204 when the operation stick 1105 is tilted to the predetermined angle or larger. Thus, when an inspector tilts the operation stick 1105 to the predetermined angle or larger to perform the rough positioning, the force required for moving the movable portion 1102 can be reduced.

Moreover, in the fourth embodiment and the fifth embodiment described above, the friction reduction mechanism includes the friction reduction portion which is provided on the peripheral edge portion of the spherical-surface portion 1301 formed on the inner ball 1205, 1501. Therefore, the friction reduction portion is in contact with the friction plate 1204 even when the operation stick 1105 is tilted in a freely selected direction by 360° degrees within the X-Z plane. Thus, even when an inspector tilts the operation stick 1105 in the freely selected direction to perform the rough positioning, the force required for moving the movable portion 1102 can be reduced.

### Second Modification Example

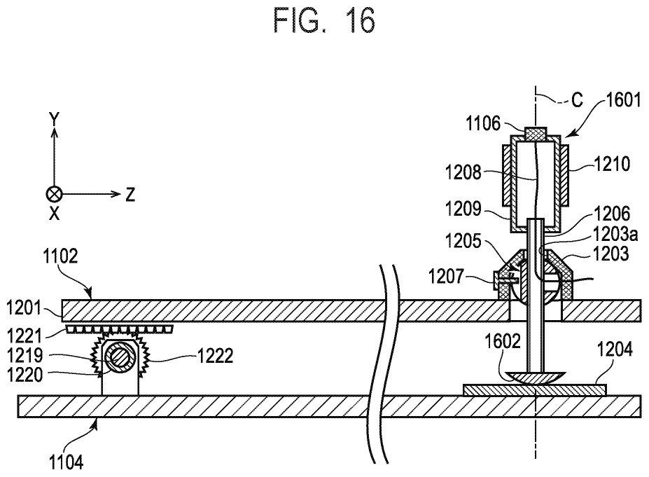

With regard to the ophthalmic apparatus 100 according to the fourth embodiment and the fifth embodiment, the case in which the inner ball 1205, 1501 is provided at the lower end portion of the operation stick 1105 and in which the inner ball 1205, 1501 and the friction plate 1204 are in contact with each other. However, the present disclosure is not limited to this case. FIG. 16 is a view for illustrating an example of a configuration of an ophthalmic apparatus of a modification example. Configurations which are the same as those of the embodiments described above are denoted by the same reference symbols, and description is omitted.

An operation stick 1601 of the second modification example includes a spherical member 1602 in addition to the inner ball 1205, the coupling shaft 1206, the lever member 1209, and the exterior cover 1210. The spherical member 1602 is provided at the lower end portion of the operation stick 1601. The spherical member 1602 corresponds to an example of the friction portion. The spherical member 1602 is fitted to the lower end of the coupling shaft 1206 penetrating the inner ball 1205. The spherical member 1602 is in contact with the upper surface of the friction plate 1204 provided on the base portion 1104. Here, when a part of the spherical member 1602 in contact with the friction plate 1204 has the same configuration as the lower portion of the inner ball 1205, 1501 of the fourth embodiment and the fifth embodiment, the same effect as the fourth embodiment and the fifth embodiment described above can be attained.

In the first to fifth embodiments and the first and second modification examples, with regard to the ophthalmic apparatus, the apparatus in which the optical head portion 101, 1101 includes the object lens portion 103, 1103, the OCT unit, the SLO unit, the anterior-eye observation unit, and the fixation lamp unit has been described. However, a configuration of the ophthalmic apparatus is not limited to such a configuration. It is only required that the ophthalmic apparatus be an apparatus to be used for measuring an eye to be inspected in a field of ophthalmology such as a freely selected OCT apparatus, a freely selected SLO apparatus, or a fundus camera apparatus.

According to the first to fifth embodiments and the first and second modification examples, at the time of rough positioning and fine positioning, the operation of positioning can be performed while consistently holding the operation stick by one hand, and at the time of rough positioning, the force required for moving the movable portion can be reduced.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-076800, filed Apr. 15, 2019, Japanese Patent Application No. 2019-169513, filed Sep. 18, 2019, and Japanese Patent Application No. 2020-055559, filed Mar. 26, 2020, which are hereby incorporated by reference herein in their entirety.

What is claimed is:

1. An ophthalmic apparatus comprising:
an optical head portion including an optical system arranged to measure an eye to be inspected;
a movable portion arranged to move the optical head portion in a horizontal direction relative to a base portion;
an operation stick, which includes an urging portion arranged to apply an urging force between the base portion and the movable portion, and is provided on the movable portion so as to be tiltable; and
an urging force reduction mechanism arranged to reduce the urging force when the operation stick is tilted by a predetermined angle or larger.

2. The ophthalmic apparatus according to claim 1, further comprising:
a friction portion provided at a lower end portion of the operation stick; and
a friction plate provided on the base portion at a position below the friction portion,
wherein the urging portion is arranged to urge the friction portion toward the friction plate, and
wherein the urging force reduction mechanism is arranged to reduce the urging force generated between the movable portion and the base portion by reducing the urging force of the friction portion against the friction plate when the operation stick is tilted by the predetermined angle or larger.

3. The ophthalmic apparatus according to claim 2,
wherein the urging force reduction mechanism includes an annular member provided to the operation stick,
wherein the annular member is brought into contact with the movable portion when the operation stick is tilted by the predetermined angle or larger, and
wherein the urging force of the friction portion against the friction plate is reduced based on a reaction force against an operation force being applied to the operation stick, which is applied from the movable portion to the operation stick when the annular member is brought into contact with the movable portion.

4. The ophthalmic apparatus according to claim 3, wherein the annular member is adjustable in position to be provided to the operation stick in an axial direction of the operation stick.

5. The ophthalmic apparatus according to claim 2,
wherein the urging force reduction mechanism includes a friction-portion receiving member provided to the movable portion,
wherein the friction portion is brought into contact with the friction-portion receiving member when the operation stick is tilted by the predetermined angle or larger,
wherein the urging force of the friction portion against the friction plate is reduced based on a reaction force against an operation force being applied to the operation stick, which is applied from the movable portion to the operation stick when the friction portion is brought into contact with the friction-portion receiving member.

6. The ophthalmic apparatus according to claim 5,
wherein the friction-portion receiving member includes an inclined surface having a symmetry axis which substantially matches a symmetry axis of the operation stick, and
wherein the friction portion is brought into contact with the inclined surface when the operation stick is tilted by the predetermined angle or larger.

7. The ophthalmic apparatus according to claim 1, wherein, when the operation stick is tilted by the predetermined angle or larger, the urging force reduction mechanism reduces the urging force based on a reaction force at the movable portion against an operation force being applied to the operation stick.

8. The ophthalmic apparatus according to claim 1, wherein, when the operation stick is tilted by the predetermined angle or larger, the urging force reduction mechanism reduces the urging force with a force in an axial direction of the operation stick generated by a reaction force at the movable portion against an operation force being applied to the operation stick.

9. The ophthalmic apparatus according to claim 1, wherein, when the operation stick is tilted by the predetermined angle or larger, the urging force reduction mechanism reduces the urging force with a force in an axial direction of the operation stick generated by an operation force being applied to the operation stick and by the movable portion.

10. The ophthalmic apparatus according to claim 1, wherein, when the operation stick is tilted by the predetermined angle or larger, the urging force reduction mechanism reduces the urging force through movement of a shaft portion of the operation stick in an axial direction relative to the movable portion so that a distance between a lower portion of the operation stick and the movable portion in the axial direction of the operation stick becomes shorter than the distance given when a tilt angle of the operation stick falls within a range up to the predetermined angle.

11. The ophthalmic apparatus according to claim 1, wherein the operation stick is tiltable to an angle which causes a force in a direction opposite to an operation force being applied to the operation stick in a tilting direction of the operation stick to act on the operation stick.

12. The ophthalmic apparatus according to claim 2,
wherein the urging force reduction mechanism includes an outer-surface portion, which has a curvature radius smaller than a curvature radius of a spherical-surface portion formed on the friction portion and is formed on a peripheral edge portion of the spherical-surface portion, and
wherein, when the operation stick is tilted to the predetermined angle or larger so that the outer-surface portion is brought into abutment against the friction plate, the friction portion separates away from the movable portion to reduce an urging force generated by the urging portion between the base portion and the movable portion.

13. The ophthalmic apparatus according to claim 12,
wherein the urging portion includes an urging member arranged to urge an inner ball, which is provided to the operation stick, and the friction portion in a longitudinal direction of the operation stick,
wherein the movable portion includes an inner-ball accommodating portion arranged to accommodate the inner ball, and
wherein the spherical-surface portion of the friction portion has a curvature radius which is substantially equal to a distance from a center of the inner ball to the friction plate.

14. The ophthalmic apparatus according to claim 13,
wherein the operation stick includes a regulation member arranged to regulate movement of the operation stick in a longitudinal direction relative to the inner ball,
wherein, when the operation stick is tilted by the predetermined angle or larger, the regulation member regulates movement of the operation stick in the longitudinal direction relative to the inner ball to regulate a distance by which the friction portion and the inner ball are separated away from each other by the urging member, and as the inner ball is separated from the inner-ball accommodating portion and the friction portion moves away from the movable portion in accordance with tilt of the operation stick, an urging force generated by the urging portion between the base portion and the movable portion is reduced.

15. The ophthalmic apparatus according to claim 14, wherein the regulation member is inserted into a hole portion formed in the inner ball and a hole portion which is formed in the operation stick and extends in the longitudinal direction of the operation stick.

16. The ophthalmic apparatus according to claim 2,
wherein the urging force reduction mechanism includes a supporting portion provided to the movable portion,
wherein, when the operation stick is tilted by the predetermined angle or larger, the supporting portion is brought into abutment against the operation stick to support the operation stick, and
wherein, when the operation stick is supported by the supporting portion so that the friction portion is brought into non-contact with the friction plate, an urging force of the friction portion against the friction plate is reduced.

17. The ophthalmic apparatus according to claim 1, further comprising:
a friction portion provided at a lower end portion of the operation stick; and
a friction plate provided on the base portion at a position below the friction portion,
wherein, when a tilt angle of the operation stick falls within a range of from 0 degrees to a predetermined angle, the movable portion allows the optical head portion to be movable in a horizontal direction relative to the base portion with the tilt of the operation stick and a friction force generated between the friction portion and the friction plate against the urging force.

18. The ophthalmic apparatus according to claim 1,
wherein the movable portion includes a first plurality of guides arranged to move the movable portion in a first direction of the horizontal direction, and a second plurality of guides arranged to move the movable portion in a second direction of the horizontal direction, the second direction intersecting with the first direction.

19. An ophthalmic apparatus, comprising:
an optical head portion including an optical system arranged to measure an eye to be inspected;
a movable portion arranged to move the optical head portion in a horizontal direction relative to a base portion; and
an operation stick provided on the movable portion so as to be tiltable,
wherein, when the operation stick is tilted by a predetermined angle or larger, a distance between a lower portion of the operation stick and the movable portion in an axial direction of the operation stick becomes shorter than the distance given when a tilt angle of the operation stick falls within a range up to the predetermined angle.

20. An ophthalmic apparatus comprising:
an optical head portion including an optical system arranged to measure an eye to be inspected;
a movable portion arranged to support the optical head portion and move the optical head portion in a horizontal direction relative to a base portion;
an operation stick provided on the movable portion so as to be tiltable;
an inner ball provided at a lower end portion of the operation stick and accommodated by the movable portion;
a friction plate held in contact with the inner ball; and
a friction reduction mechanism arranged to reduce a friction coefficient between the inner ball and the friction plate when the operation stick is tilted to a predetermined angle or larger.

21. The ophthalmic apparatus according to claim 20,
wherein the friction reduction mechanism includes a friction reduction portion which is provided on a peripheral edge portion of a spherical-surface portion formed on the inner ball, and
wherein, when the operation stick is tilted to the predetermined angle or larger to bring the friction reduction portion into contact with the friction plate, the friction coefficient between the inner ball and the friction plate is reduced.

22. The ophthalmic apparatus according to claim 21, wherein the friction reduction portion is an outer-surface portion, which has a surface roughness smaller than a surface roughness of the spherical-surface portion and is provided on the peripheral edge portion of the spherical-surface portion.

23. The ophthalmic apparatus according to claim 22, wherein the spherical-surface portion and the outer-surface portion form parts of the same spherical shape and are provided in different regions on the spherical shape.

24. The ophthalmic apparatus according to claim 22, wherein a boundary line between the spherical-surface portion and the outer-surface portion has a circular shape as seen from an axis direction of the operation stick.

25. The ophthalmic apparatus according to claim 21, wherein the friction reduction portion is a rolling element retained on the peripheral edge portion of the spherical-surface portion.

26. The ophthalmic apparatus according to claim 25, wherein the rolling element comprises a plurality of rolling elements which are arranged at intervals along the peripheral edge portion of the spherical-surface portion as seen from the axis direction of the operation stick.
